(19) United States
(12) Patent Application Publication
(10) Pub. No.: US 2026/0125748 A1
(43) Pub. Date: May 7, 2026

(54) METHYLATION SEQUENCING THROUGH METHYLATION PRESERVING AMPLIFICATION WITH ERROR CORRECTION

(71) Applicant: GUARDANT HEALTH, INC., Palo Alto, CA (US)

(72) Inventor: Andrew KENNEDY, San Diego, CA (US)

(21) Appl. No.: 19/243,171

(22) Filed: Jun. 19, 2025

Related U.S. Application Data
(63) Continuation of application No. PCT/US2023/085248, filed on Dec. 20, 2023.
(60) Provisional application No. 63/476,914, filed on Dec. 22, 2022.

(51) Int. Cl. C12Q 1/6848; C12Q 1/48; C12Q 1/6806; C12Q 1/6855; C12Q 1/6869; C12Q 1/6876

(52) U.S. Cl. CPC C12Q 1/6848; C12Q 1/48; C12Q 1/6806; C12Q 1/6855; C12Q 1/6869; C12Q 1/6876; C12Y 201/01; C12Q 2600/154

(57) ABSTRACT

Provided herein is a DNA analysis method comprising methylation-specific amplification with error correction. Provided herein are also methods for determining the likelihood that a subject has a disease or condition, such as cancer.

### CROSS-REFERENCE TO RELATED APPLICATIONS

The present application is a Continuation of International Patent Application No. PCT/US2023/085248, filed Dec. 20, 2023, which claims the benefit of priority of U.S. Provisional Patent Application No. 63/476,914, filed Dec. 22, 2022, which is incorporated by reference herein for all purposes.

### FIELD OF THE INVENTION

The present disclosure provides compositions and methods related to analyzing DNA, such as cell-free DNA. In some embodiments, the DNA is from a subject having or suspected of having cancer and/or the DNA includes DNA from cancer cells. In some embodiments, the DNA is amplified using a methylation-preserving amplification, prior to sequencing. In some embodiments, the methylation-preserving amplification comprises a DNA methyltransferase, such as DNA methyltransferase 1 (DNMT1).

### INTRODUCTION AND SUMMARY

Cancer is responsible for millions of deaths per year worldwide. Early detection of cancer may result in improved outcomes because early-stage cancer tends to be more susceptible to treatment.

Improperly controlled cell growth is a hallmark of cancer that generally results from an accumulation of genetic and epigenetic changes, such as copy number variations (CNVs), single nucleotide variations (SNVs), gene fusions, insertions and/or deletions (indels), epigenetic variations including modification of cytosine (e.g., 5-methylcytosine, 5-hydroxymethylcytosine, and other more oxidized forms) and association of DNA with chromatin proteins and transcription factors. Thus, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. Hypermethylation can be associated with an aberrant loss of transcriptional capacity of involved genes and occurs at least as frequently as point mutations and deletions as a cause of altered gene expression. Furthermore, without wishing to be bound by any particular theory, cells in or around a cancer or neoplasm may shed more DNA than cells of the same tissue type in a healthy subject. The DNA from such cells may differ epigenetically from shed DNA in a healthy subject. As such, the distribution of epigenetically modified (e.g., methylated) DNA in certain DNA samples, such as cell-free DNA (cfDNA), may change upon carcinogenesis. Thus, sufficiently sensitive epigenetic (e.g., DNA methylation) profiling can be used to detect aberrant methylation in DNA of a sample.

Biopsies represent a traditional approach for detecting or diagnosing cancer in which cells or tissue are extracted from a possible site of cancer and analyzed for relevant phenotypic and/or genotypic features. Biopsies have the drawback of being invasive.

Detection of cancer based on analysis of body fluids (“liquid biopsies”), such as blood, is an intriguing alternative based on the observation that DNA from cancer cells is released into body fluids. A liquid biopsy is noninvasive (sometimes requiring only a blood draw). However, it has been challenging to develop accurate and sensitive methods for analyzing liquid biopsy material that provides detailed information regarding nucleobase modifications given the low concentration and heterogeneity of cell-free DNA. The contribution of DNA from cells in or around a cancer or neoplasm to a sample may be relatively small relative to the contribution from other cells, and the DNA contributed from other cells may be uninformative as to cancer status. Isolating and processing the fractions of cell-free DNA useful for further analysis in liquid biopsy procedures is an important part of these methods.

Further, current methods of cancer diagnostic assays of cell-free nucleic acids (e.g., cell-free DNA or cell-free RNA) may focus on the detection of tumor-related somatic variants, including single nucleotide variants (SNVs), copy number variations (CNVs), fusions, and indels (i.e., insertions or deletions), which are all mainstream targets for liquid biopsy. There is growing evidence that non-sequence modifications like methylation status and fragmentomic signal in cell-free DNA can provide information on the source of cell-free DNA and disease level. In addition, different types of modifications such as 5-methylation and 5-hydroxymethylation can have different implications as to the presence or absence of disease. Detailed knowledge of the non-sequence modifications of the cell-free DNA (e.g., when combined with somatic mutation calling) can improve assessments of tumor status.

Accordingly, there is a continued need for improved methods and compositions for analyzing DNA, including cell-free DNA, e.g., in liquid biopsies.

The present disclosure aims to meet the need for improved analysis of DNA, such as cell-free DNA and/or provide other benefits. In some embodiments, the present disclosure provides single-base resolution sequencing methods that exhibit increased sensitivity (e.g., ability to detect epigenetic features reliably in samples containing fewer molecules) and/or reduced molecular losses compared to other sequencing methods, such as compared to methods that include a pre-amplification epigenetic base conversion step.

The following exemplary embodiments are provided.

Embodiment 1 is a method of analyzing DNA, the method comprising:
(a) performing a methylation-preserving amplification of the DNA, wherein the DNA comprises barcodes; and
(b) sequencing the DNA in a modification-sensitive manner and determining an epigenetic consensus sequence of the DNA associated with at least a portion of the barcodes.

Embodiment 2 is a method of analyzing DNA, the method comprising:
(a) performing a methylation-preserving amplification of the DNA, wherein the DNA comprises barcodes;
(b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and
(c) sequencing the DNA and determining an epigenetic consensus sequence of the DNA associated with at least a portion of the barcodes.

Embodiment 3 is a method of analyzing DNA, the method comprising:
(a) performing a methylation-preserving amplification of the DNA, wherein the DNA comprises barcodes;
(b) enriching for one or more sets of epigenetic target regions of DNA from the DNA, thereby providing enriched DNA;
(c) sequencing the enriched DNA in a modification-sensitive manner and determining an epigenetic consensus sequence of the enriched DNA associated with at least a portion of the barcodes.

Embodiment 4 is a method of analyzing DNA, the method comprising:
(a) performing a linear, methylation-preserving amplification of the DNA; and
(b) sequencing the DNA in a modification-sensitive manner and determining an epigenetic consensus sequence of the DNA.

Embodiment 5 is a method of analyzing DNA, the method comprising:
(a) performing a linear, methylation-preserving amplification of the DNA;
(b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and
(c) sequencing the DNA and determining an epigenetic consensus sequence of the DNA.

Embodiment 6 is a method of analyzing DNA, the method comprising:
(a) performing a linear, methylation-preserving amplification of the DNA;
(b) enriching for one or more sets of epigenetic target regions of DNA from the DNA, thereby providing enriched DNA;
(c) sequencing the enriched DNA in a modification-sensitive manner and determining an epigenetic consensus sequence of the enriched DNA.

Embodiment 7 is the method of any one of embodiments 1, 3, 4, or 6, wherein the sequencing in a modification-sensitive manner comprises subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

Embodiment 8 is the method of any one of the preceding embodiments, wherein the DNA comprises barcodes.

Embodiment 8.1 is the method of embodiment 8, wherein the barcodes are non-unique barcodes.

Embodiment 9 is the method of any one of the preceding embodiments, wherein the method comprises ligating adapters comprising barcodes to the DNA prior to the sequencing.

Embodiment 10 is the method of any one of the preceding embodiments, wherein the method comprises ligating adapters comprising barcodes to the DNA prior to amplifying the DNA.

Embodiment 11 is the method of any one of the preceding embodiments, wherein the method comprises ligating adapters comprising barcodes to the DNA prior to performing the methylation-preserving amplification of the DNA.

Embodiment 12 is a method of analyzing DNA, the method comprising:
(a) performing a methylation-preserving amplification of the DNA, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site, thereby providing amplified DNA;
(b) contacting the amplified DNA with a restriction enzyme that recognizes and cleaves the DNA at the restriction enzyme cleavage site in the adapter;
(c) before or after step (b), subjecting the amplified DNA to a procedure that affects a first nucleobase of the amplified DNA differently from a second nucleobase of the amplified DNA, wherein the first nucleobase is a modified or unmodified nucleobase, the second nucleobase is a modified or unmodified nucleobase different from the first nucleobase, and the first nucleobase and the second nucleobase have the same base pairing specificity;
(d) after steps (b) and (c), ligating supplemental adapters to the amplified DNA; (e) after step (d), performing a uracil- and/or dihydrouracil-tolerant amplification of the DNA;
(f) after step (e), enriching for one or more sets of epigenetic target regions of DNA from the amplified DNA, thereby providing enriched DNA;
(g) optionally further amplifying the enriched DNA; and
(h) sequencing the enriched DNA and determining an epigenetic consensus sequence of the DNA associated with at least a portion of the barcodes.

Embodiment 13 is the method of embodiment 12, wherein the uracil- and/or dihydrouracil-tolerant amplification of the DNA comprises PCR using a uracil- and/or dihydrouracil-tolerant DNA polymerase.

Embodiment 14 is the method of embodiment 12 or embodiment 13, wherein the optional step of amplifying the enriched DNA (step (g)) is performed.

Embodiment 15 is the method of embodiment 14, wherein the step of amplifying the enriched DNA further comprises differentially tagging the enriched DNA.

Embodiment 16 is the method of embodiment 15, wherein differentially tagging the enriched DNA comprises attaching one or more sample indices to the DNA.

Embodiment 17 is the method of any one of embodiments 1-3 or 7-16, wherein the barcodes do not comprise cytosines in a non-CpG context.

Embodiment 18 is the method of any one of embodiments 1-10 or 17, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site.

Embodiment 18.1 is the method of any one of the preceding embodiments, wherein the barcodes are molecular barcodes, wherein the molecular barcodes distinguish different DNA molecules in the same sample.

Embodiment 18.2 is the method of embodiment 18.1, wherein the molecular barcodes were added to the DNA molecules, optionally wherein the molecular barcodes were added by ligation.

Embodiment 19 is the method of any one of embodiments 18-18.2, further comprising contacting the amplified DNA with a restriction enzyme that recognizes and cleaves the DNA at the restriction enzyme cleavage site in the adapter.

Embodiment 20 is the method of embodiment 19, further comprising ligating supplemental adapters to the amplified DNA (a) after the step of contacting the amplified DNA with a restriction enzyme that recognizes and cleaves the DNA at the restriction enzyme cleavage site in the adapter, and (b) prior to or after a step of subjecting the amplified DNA to a procedure that affects a first nucleobase of the amplified DNA differently from a second nucleobase of the amplified DNA, wherein the first nucleobase is a modified or unmodified nucleobase, the second nucleobase is a modified or unmodified nucleobase different from the first nucleobase, and the first nucleobase and the second nucleobase have the same base pairing specificity.

Embodiment 21 is the method of embodiment 20, further comprising performing a uracil- and/or dihydrouracil-tolerant amplification of the DNA.

Embodiment 22 is the method of embodiment 21, wherein the uracil- and/or dihydrouracil-tolerant amplification of the DNA comprises PCR using a uracil- and/or dihydrouracil-tolerant DNA polymerase.

Embodiment 23 is the method of any one of embodiments 11-17 or 20-22, wherein the supplemental adapters do not comprise barcodes.

Embodiment 24 is the method of any one of embodiments 11-17 or 19-23, wherein cleavage of the DNA by the restriction enzyme results in an overhang.

Embodiment 25 is the method of embodiment 24, wherein the overhang is a single base overhang.

Embodiment 26 is the method of embodiment 25, wherein the single base overhang is a single base 5′-overhang.

Embodiment 27 is the method of embodiment 25 or embodiment 26, wherein the single base overhang is a T.

Embodiment 28 is the method of embodiment 25 or embodiment 26, wherein the single base overhang is an A.

Embodiment 29 is the method of any one of the preceding embodiments, wherein the methylation-preserving amplification comprises contacting the DNA with a methyltransferase.

Embodiment 30 is the method of embodiment 29, wherein the methyltransferase preferentially methylates hemimethylated CpG and/or hemimethylated CpHpG.

Embodiment 31 is the method of embodiment 29 or embodiment 30, wherein the methyltransferase is DNMT1.

Embodiment 32 is the method of any one of the preceding embodiments, wherein the methylation-preserving amplifying comprises one or more of polymerase chain reaction, linear amplification, rolling circle amplification, ligase chain reaction, strand displacement amplification, nucleic acid sequence-based amplification, and self-sustained sequence-based replication.

Embodiment 33 is the method of any one of the preceding embodiments, wherein the methylation-preserving amplification comprises thermocycled amplification.

Embodiment 34 is the method of any one of the preceding embodiments, wherein the methylation-preserving amplification comprises isothermal amplification.

Embodiment 35 is the method of embodiment 34, wherein the isothermal amplification comprises recombinase polymerase amplification (RPA), helices dependent amplification (HDA), loop-mediated isothermal amplification (LAMP), or rolling circle amplification (RCA).

Embodiment 36 is the method of any one of the preceding embodiments, wherein the methylation-preserving amplification comprises linear amplification with thermocycling.

Embodiment 37 is the method of any one of embodiments 2, 5, or 7-36, wherein the first nucleobase is an unmodified cytosine and the second nucleobase is a modified cytosine, optionally wherein the modified cytosine is 5-methylcytosine or 5-hydroxymethylcytosine.

Embodiment 38 is the method of any one of embodiments 2, 5, or 7-37, wherein the procedure that affects a first nucleobase of the DNA differently from a second nucleobase of the DNA is performed:
before the enriching or after the enriching; and
before the sequencing.

Embodiment 39 is the method of any one of embodiments 2, 5, or 7-38, wherein the procedure that affects a first nucleobase of the DNA differently from a second nucleobase of the DNA chemically converts the first or second nucleobase such that the base pairing specificity of the converted nucleobase is altered.

Embodiment 40 is the method of any one of embodiments 2, 5, or 7-39, wherein the procedure that affects a first nucleobase of the DNA differently from a second nucleobase of the DNA is methylation-sensitive conversion.

Embodiment 41 is the method of embodiment 40, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, or enzymatic conversion.

Embodiment 42 is the method of embodiment 41, wherein the Tet-assisted conversion further comprises a substituted borane reducing agent, optionally wherein the substituted borane reducing agent is 2-picoline borane, borane pyridine, tert-butylamine borane, or ammonia borane.

Embodiment 43 is the method of any one of the preceding embodiments, wherein the method further comprises partitioning at least a portion of the DNA into a plurality of subsamples, comprising a first subsample and a second subsample, wherein the first subsample comprises DNA with a cytosine modification in a greater proportion than the second subsample.

Embodiment 44 is the method of embodiment 43, wherein the partitioning at least a portion of the DNA into a plurality of subsamples comprises contacting the DNA with an agent that recognizes a modified cytosine in the DNA, wherein the first subsample comprises DNA with the modified cytosine in a greater proportion than the second subsample.

Embodiment 45 is the method of embodiment 43 or embodiment 44, wherein the partitioning is performed prior to the sequencing and
i. prior to performing the methylation-preserving amplification of the DNA,
ii. after performing the methylation-preserving amplification of the DNA,
iii. prior to the enriching for one or more sets of epigenetic target regions of DNA from the DNA; and/or
iv. after the enriching for one or more sets of epigenetic target regions of DNA from the DNA.

Embodiment 46 is the method of embodiment 44 or embodiment 45, wherein the agent that recognizes a modified nucleobase in the DNA is a methyl binding reagent.

Embodiment 47 is the method of embodiment 46, wherein the methyl binding reagent is a methyl binding domain (MBD) protein or an antibody.

Embodiment 48 is the method of embodiment 46 or embodiment 47, wherein the methyl binding reagent is specific to one or more methylated nucleotide bases, optionally wherein the one or more methylated nucleotide bases is 5-methylcytosine.

Embodiment 49 is the method of any one of embodiments 46-48, wherein the methyl binding reagent is immobilized on a solid support.

Embodiment 50 is the method of any one of embodiments 46-49, wherein the partitioning comprises immunoprecipitation of methylated DNA.

Embodiment 51 is the method of any one of embodiments 46-50, wherein the partitioning comprises partitioning on the basis of binding to a protein, optionally wherein the protein is a methylated protein, an acetylated protein, an unmethylated protein, an unacetylated protein; and/or optionally wherein the protein is a histone.

Embodiment 52 is the method of any one of embodiments 46-51, wherein the partitioning comprises contacting the DNA with a binding reagent which is specific for the protein and is immobilized on a solid support.

Embodiment 53 is the method of any one of embodiments 46-52, wherein a first partitioned subsample of the plurality of partitioned subsamples is differentially tagged from a second partitioned subsample of the plurality of partitioned subsamples.

Embodiment 54 is the method of any one of the preceding embodiments, comprising contacting the DNA or at least one subsample thereof with at least one nuclease prior to the enriching or prior to the sequencing, optionally wherein the at least one nuclease is at least one restriction enzyme.

Embodiment 55 is the method of embodiment 54, wherein the contacting the DNA or at least one subsample thereof with at least one nuclease occurs after partitioning the sample into the plurality of subsamples or before the performing a procedure that affects a first nucleobase of the DNA differently from a second nucleobase of the DNA.

Embodiment 56 is the method of embodiment 54 or embodiment 55, wherein the at least one restriction enzyme is a methylation-sensitive restriction enzyme (MSRE).

Embodiment 57 is the method of embodiment 54 or embodiment 55, wherein the at least one restriction enzyme is a methylation-dependent restriction enzyme (MDRE).

Embodiment 58 is the method of any one of embodiments 54-56, wherein the DNA or the at least one subsample thereof is contacted with the at least one methylation sensitive restriction enzyme, thereby producing hypermethylated DNA.

Embodiment 59 is the method of any one of embodiments 54, 55, or 57, wherein the DNA or the at least one subsample thereof is contacted with the at least one methylation dependent restriction enzyme, thereby producing hypomethylated DNA.

Embodiment 60 is the method of any one of embodiments 54-56, 58, or 59, wherein the first subsample is contacted with the MSRE.

Embodiment 61 is the method of any one of embodiments 54, 55, or 57-60, wherein the second subsample is contacted with the MDRE.

Embodiment 62 is the method of any one of embodiments 54-61, comprising contacting at least one subsample with at least two restriction enzymes prior to the enriching or sequencing, optionally wherein the contacting occurs before performing the procedure that affects a first nucleobase of the DNA differently from a second nucleobase of the DNA.

Embodiment 63 is the method of embodiment 62, wherein the at least two restriction enzymes comprise or consist of two or three restriction enzymes.

Embodiment 64 is the method of any one of embodiments 54-63, wherein the at least one restriction enzyme is selected from the group consisting of FspEI, LpnPI, MspJI, SgeI. AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI.

Embodiment 65 is the method of any one of embodiments 54-64, further comprising, prior to the digesting, attaching one or more adapters to at least one end of at least a portion of the DNA molecules in the plurality of partitioned sets.

Embodiment 66 is the method of embodiment 65, wherein the one or more adapters comprises at least one tag.

Embodiment 67 is the method of embodiment 66, wherein the at least one tag comprises a molecular barcode.

Embodiment 68 is the method of any one of embodiments 65-67, wherein the one or more adapters is resistant to digestion by methylation sensitive restriction enzymes or methylation dependent restriction enzymes.

Embodiment 69 is the method of embodiment 68, wherein the one or more adapters that is resistant to digestion by methylation sensitive restriction enzymes comprises
a) one or more methylated nucleotides, optionally wherein the methylated nucleotides comprise 5-methylcytosine and/or 5-hydroxymethylcytosine;
b) one or more nucleotide analogs resistant to methylation sensitive restriction enzymes; or
c) a nucleotide sequence not recognized by methylation sensitive restriction enzymes.

Embodiment 70 is the method of any one of embodiments 43-69, wherein the subsamples are pooled prior to the sequencing.

Embodiment 71 is the method of any one of embodiments 1, 2, 4, 5, 7-10, or 17-70, further comprising enriching for one or more sets of epigenetic target regions of DNA from the DNA, thereby providing enriched DNA.

Embodiment 72 is the method of any one of the preceding embodiments, further comprising enriching for one or more sets of sequence-variable target regions of DNA from the DNA.

Embodiment 73 is the method of any one of the preceding embodiments, wherein the enriching comprises contacting the DNA with target-specific probes specific for the one or more sets of epigenetic target regions and/or for the one or more sets of sequence-variable target regions.

Embodiment 74 is the method of any one of embodiments 3 or 6-73, wherein the epigenetic target region set comprises a hypermethylation variable target region set and/or a hypomethylation variable target region set.

Embodiment 75 is the method of any one of embodiments 3 or 6-74, wherein the epigenetic target region set comprise a fragmentation variable target region set.

Embodiment 76 is the method of embodiment 75, wherein the fragmentation variable target region set comprises transcription start site regions.

Embodiment 77 is the method of embodiment 75 or embodiment 76, wherein the fragmentation variable target region set comprises CTCF binding regions.

Embodiment 78 is the method of any one of embodiments 3 or 6-77, wherein the epigenetic target region set comprises one or more type-specific epigenetic target regions.

Embodiment 79 is the method of embodiment 78, wherein the one or more type-specific epigenetic target regions comprises type-specific differentially methylated regions and/or type specific fragments.

Embodiment 80 is the method of embodiment 78, wherein the one or more type-specific epigenetic target regions comprises type-specific hypomethylated regions and/or type-specific hypermethylated regions.

Embodiment 81 is the method of any one of embodiments 78-80, wherein the one or more type-specific epigenetic target regions comprises cell-type specific, cell cluster-type specific, tissue-type specific, and/or cancer-type specific epigenetic target regions.

Embodiment 82 is the method of any one of embodiments 78-81, wherein the one or more type-specific epigenetic target regions comprise target regions that are:
a) hypermethylated in immune cells relative to non-immune cell types present in a blood sample;
b) differentially methylated in colon relative to other tissue types;
c) differentially methylated in breast relative to other tissue types;
d) differentially methylated in liver relative to other tissue types;
e) differentially methylated in kidney relative to other tissue types;
f) differentially methylated in pancreas relative to other tissue types;
g) differentially methylated in prostate relative to other tissue types;
h) differentially methylated in skin relative to other tissue types; or
i) differentially methylated in bladder relative to other tissue types.

Embodiment 83 is the method of any one of embodiments 78-82, wherein the hypermethylated target regions are methylated to an extent that is at least 10%, 20%, 30%, or at least 40% greater than the average methylation of the target regions in the sample or relative to other cell or tissue types.

Embodiment 84 is the method of any one of embodiments 78-83, wherein the one or more type-specific epigenetic target regions comprises
a) target regions that are hypomethylated in non-immune cell types present in the sample relative to the methylation level of the target regions in a different cell or tissue type in the sample;
b) fragments specific to immune cells relative to non-immune cell types present in the sample; or
c) fragments specific to colon, lung, breast, liver, kidney, pancreas, prostate, skin, or bladder relative to other tissue types.

Embodiment 85 is the method of any one of embodiments 78-84, wherein the level of the one or more type-specific epigenetic target regions that originated from a cell type or a tissue type is determined.

Embodiment 86 is the method of embodiment 78-85, wherein the levels of the one or more type-specific epigenetic target regions that originated from one or more immune cells, non-immune cell types present in a blood sample, and/or colon, lung, breast, liver, kidney, prostate, skin, bladder, or pancreas cells are determined.

Embodiment 87 is the method of any one of embodiments 78-86, further comprising identifying at least one cell type, cell cluster type, tissue type, and/or cancer type from which the one or more type-specific epigenetic target regions originated.

Embodiment 88 is the method of any one of embodiments 78-87, comprising determining the methylation levels of the type-specific epigenetic target regions.

Embodiment 89 is the method of any one of embodiments 43-88, wherein at least a portion of the DNA from the first subsample and at least a portion of the DNA from the second subsample are pooled, thereby providing a combined subsample.

Embodiment 90 is the method of any one of embodiments 43-89, wherein the DNA of the first subsample and the DNA of the second subsample are differentially tagged.

Embodiment 91 is the method of the immediately preceding embodiment, wherein the pool comprises less than or equal to about 45%, 40%, 35%, 30%, 25%, 20%, 15%, 10%, or 5% of the DNA of the second subsample.

Embodiment 92 is the method of embodiment 90 or embodiment 91, wherein the pool comprises about 70-90%, about 75-85%, or about 80% of the DNA of the second subsample.

Embodiment 93 is the method of any one of embodiments 90-92, wherein the pool comprises substantially all of the DNA of the first subsample.

Embodiment 94 is the method of any one of embodiments 90-92, wherein the pool comprises substantially all of the DNA of the first subsample or treated first subsample.

Embodiment 95 is the method of any one of embodiments 90-94, wherein the first target region set is captured from at least a portion of the first subsample after formation of the pool.

Embodiment 96 is the method of any one of embodiments 90-95, wherein at least a portion of the DNA from the first subsample and at least a portion of the DNA from the second subsample are sequenced in the same sequencing cell.

Embodiment 97 is the method of any one of embodiments 90-96, wherein the plurality of subsamples comprises a third subsample, which comprises DNA with the epigenetic modification in a greater proportion than the second subsample but in a lesser proportion than the first subsample.

Embodiment 98 is the method of the immediately preceding embodiment, wherein the method further comprises differentially tagging the third subsample.

Embodiment 99 is the method of embodiment 97 or embodiment 98, wherein DNA from the first subsample, DNA from the third sample, and the target region set are pooled, optionally wherein DNA from the first, second, and third subsamples is sequenced in the same sequencing cell.

Embodiment 100 is the method of any one of embodiments 89-99, wherein the sequencing the DNA of the combined subsample comprises sequencing the DNA in a modification-sensitive manner.

Embodiment 101 is the method of any one of embodiments 3, 6-10, or 17-96, wherein the DNA is amplified after the enriching step.

Embodiment 102 is the method of embodiment 101, wherein the amplifying further comprises differentially tagging the enriched DNA.

Embodiment 103 is the method of embodiment 102, wherein differentially tagging the enriched DNA comprises attaching one or more sample indices to the DNA.

Embodiment 104 is the method of any one of the preceding embodiments, wherein the sequencing the DNA comprises sequencing the DNA in a manner that distinguishes the first nucleobase from the second nucleobase.

Embodiment 105 is the method of any one of embodiments 1, 3, 4, or 6-104, wherein the sequencing in a modification-sensitive manner comprises long-read sequencing.

Embodiment 106 is the method of any one of embodiments 1, 3, 4, or 6-105, wherein the sequencing in a modification-sensitive manner comprises nanopore sequencing.

Embodiment 107 is the method of any one of embodiments 1, 3, 4, or 6-104, wherein the sequencing in a modification-sensitive manner comprises 5-letter or 6-letter sequencing.

Embodiment 108 is the method of any one of the preceding embodiments, wherein the sequencing comprises next generation sequencing.

Embodiment 109 is the method of any one of the preceding embodiments, wherein the sequencing comprises generating a plurality of sequencing reads; and the method further comprises mapping the plurality of sequence reads to one or more reference sequences to generate mapped sequence reads, and processing the mapped sequence reads corresponding to the sequence-variable target region set and to the epigenetic target region set.

Embodiment 110 is the method of any one of embodiments 4-109, further comprising determining an epigenetic consensus sequence of the DNA associated with at least a portion of the barcodes.

Embodiment 111 is the method of any one of embodiments 1-3, 7-10, or 17-110, wherein the determining an epigenetic consensus sequence of the DNA associated with at least a portion of the barcodes comprises comparing the sequences of at least a portion of reads associated with (a) a unique barcode or a unique set of barcodes, (b) a unique genomic start and/or stop position, or (c) both (a) and (b); wherein the reads originated from a DNA molecule; and determining a consensus epigenetic status of each nucleotide of the DNA molecule.

Embodiment 112 is the method of any one of embodiments 1-3, 7-10, or 17-111, further comprising determining a consensus base sequence of the DNA associated with at least a portion of the barcodes.

Embodiment 113 is the method of embodiment 112, wherein the determining a consensus base sequence of the DNA associated with at least a portion of the barcodes comprises comparing the sequences of at least a portion of the reads associated with (a) a unique barcode or a unique set of barcodes, (b) a unique genomic start and/or stop position, or (c) both (a) and (b); wherein the reads originated from a DNA molecule;
and determining a consensus base identity of each nucleotide of the DNA molecule.

Embodiment 114 is the method of any one of the preceding embodiments, wherein the DNA is cell-free DNA.

Embodiment 115 is the method of embodiment 114, wherein the cell-free DNA is in an amount between 1 ng and 500 ng.

Embodiment 116 is the method of any one of the preceding embodiments, wherein the DNA is from a blood sample and/or a tissue sample.

Embodiment 117 is the method of embodiment 116, wherein the blood sample is a whole blood sample, a plasma sample, a buffy coat sample, a leukapheresis sample, or a PBMC sample.

Embodiment 118 is the method of any one of the preceding embodiments, wherein the DNA and/or the sample is from a subject.

Embodiment 119 is the method of embodiment 118, wherein the subject is an animal.

Embodiment 120 is the method of embodiment 118 or embodiment 119, wherein the subject is a human.

Embodiment 121 is the method of any one of embodiments 94-120, wherein the blood sample is fractionated prior to enriching for at least one epigenetic target region sets of DNA.

Embodiment 122 is the method of any one of embodiments 118-121, wherein the subject has or is at risk of having a cancer.

Embodiment 123 is the method of embodiment 118-122, further comprising determining the presence or status of a cancer in the subject.

Embodiment 124 is the method of any one of embodiments 118-123, further comprising determining the likelihood that the subject has an infection.

Embodiment 125 is the method of any one of embodiments 118-124, further comprising determining the likelihood that the subject has a transplant rejection.

### BRIEF DESCRIPTION OF THE DRAWINGS

FIG. 1A illustrates exemplary workflows according to certain embodiments disclosed herein.

FIG. 1B illustrates exemplary workflows according to certain embodiments disclosed herein.

FIG. 2 is a schematic diagram of an example of a system suitable for use with some embodiments of the disclosure.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

As used herein, “amplify,” “amplifying,” or “amplification” refers to a process by which extra or multiple copies of a particular polynucleotide are formed. Amplification methods can include any suitable methods known in the art. As used herein, a nucleic acid molecule amplified using “methylation-preserving amplification” substantially maintains its methylation status post-amplification.

“Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, a “methyltransferase” or a “DNA methyltransferase” refers to any enzyme that methylates DNA, e.g., at the 5-carbon of cytosine, forming 5′methylcytosine, e.g., DNA methyltransferases of Enzyme Commission number (EC) 2.1.1.37. DNA methyltransferases that can be used in the methods described herein include methyltransferases (including modified, evolved, or engineered methyltransferases) that can act on hemimethylated CpG or hemimethylated CpHpG (where H=A, C, or T). Such DNA methyltransferases can include, but are not limited to, DNMT1 and a modified DNMT3 that acts on hemimethylated CpG or hemimethylated CpHpG. These enzymes can use S-adenosylmethionine as a methyl donor. Additional examples of methyltransferases are described elsewhere herein.

“DNA methyltransferase 1” or “DNMT1” refers to an enzyme (UniProt Accession No. K7EP77) encoded by a DNMT1 gene. This enzyme transfers methyl groups to cytosine nucleotides of genomic DNA, preferentially methylating hemi-methylated DNA, particularly the unmethylated cytosine of a hemi-methylated CpG dinucleotide. DNMT1 is an enzyme primarily responsible for maintaining methylation patterns following DNA replication.

As used herein, “fragment” refers to a biological component, such as a nucleic acid molecule (such as DNA or RNA) that has been broken or separated from one or more other pieces. Fragmentation, such as DNA fragmentation, can occur spontaneously (as in cfDNA fragments, which may be obtained from blood samples) or can be induced intentionally, such as using standard laboratory procedures, such as described herein. DNA fragmentation can be performed, for example, to prepare DNA (such as genomic DNA and/or DNA isolated from a sample comprising cells) for sequencing. With some samples, such as cfDNA samples, artificial fragmentation may be unnecessary.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

“Enriching” or “capturing” one or more target nucleic acids or one or more nucleic acids comprising at least one target region refers to preferentially isolating or separating the one or more target nucleic acids or one or more nucleic acids comprising at least one target region from non-target nucleic acids or from nucleic acids that do not comprise at least one target region.

An “enriched set” or “captured set” of nucleic acids or “enriched” or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “cell type” is a set of cells having a shared characteristic. For example, cell types can include cells of different origins, differentiation types, different activation types, or any combination of different origins, different differentiation types, and different activation types. Indeed, differentiation status and activation status can overlap and often change together in a given cell, such as an immunce cell or a cancer cell. For example, activation of an immune cell may induce differentiation of the cell. In some embodiments, cell types may be distinguished based on characteristics such as one or more cell surface markers, a genetic signature (such as expression (or expression level) of a particular gene or set of genes), and/or an epigenetic signature, such as regions of DNA hypermethylation or hypomethylation.

As used herein, a “cell cluster” or “cluster” is a plurality of related cell types, e.g., immune cell types, tissue-specific cell types, and/or cancer cell types. In some embodiments, the cell types within a cluster have similar DNA methylation profiles, e.g., in a plurality of hypermethylation variable target regions and/or hypomethylation variable target regions.

A “converted nucleobase” is a nucleobase having an altered base pairing specificity, wherein the original base pairing specificity of the nucleobase was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. As used herein, a “converted sample” is a sample comprising DNA comprising at least one converted nucleobase.

As used herein, a “combination” of steps or other elements refers to the performance or presence of two or more of the steps or elements in a method or product; elements, where appropriate, may be either together in a single composition, apparatus, or the like, or in proximity, e.g., in separate containers or compartments within a larger container, such as a multiwell plate, tube rack, refrigerator, freezer, incubator, water bath, ice bucket, machine, or other form of storage. A combination, combinations, or combination thereof refers to any and all permutations and combinations of the listed terms preceding the term “combination.” For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Specifically binds” in the context of a primer, a probe, or other oligonucleotide and a target sequence (e.g., a nucleic acid comprising a sequence that is partially or completely complementary to the primer, probe, or other oligonucleotide) means that under appropriate hybridization conditions, the primer, probe, or other oligonucleotide hybridizes to its target sequence, or replicates thereof, to form a stable hybrid, while at the same time formation of stable non-target hybrids is minimized. Thus, a primer, probe, or other oligonucleotide hybridizes to a target sequence or replicate thereof to a sufficiently greater extent than to a non-target sequence, to ultimately enable enrichment or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

A “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity). A “target region set” can comprise regions that share at least one common feature. In some embodiments, a target region set is identified by the at least one common feature. For example, a hypermethylation variable target region set comprises regions of DNA that are hypermethylated.

“Sequence-variable target regions” refer to target regions that may exhibit changes in sequence such as nucleotide substitutions (i.e., single nucleotide variations), insertions, deletions, or gene fusions, or transpositions in neoplastic cells (e.g., tumor cells and cancer cells) relative to normal cells. A “sequence-variable target region set” refers to a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, 2, or 1 nucleotides.

“Epigenetic target regions” refers to target regions that may show sequence-independent differences in different cell or tissue types (e.g., different types of immune cells) or in abnormal cells, such as neoplastic cells (e.g., tumor cells and cancer cells), relative to normal cells; or that may show sequence-independent differences (i.e., in which there is no change to the nucleotide sequence, e.g., differences in methylation, nucleosome distribution, or other epigenetic features) in DNA, e.g., from different cell types or from subjects having cancer relative to DNA from healthy subjects. Examples of sequence-independent changes include, but are not limited to, changes in methylation (increases or decreases), nucleosome distribution, fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An “epigenetic target region set” is a set of epigenetic target regions.

As used herein, a “differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one immune cell type, relative to the degree of methylation in the same region of DNA from at least one other cell or tissue type, such as other immune cell types, or from the same cell or tissue type from a healthy subject. In some embodiments, a differentially methylated region has a detectably lower degree of methylation (e.g., a hypomethylated region) in at least one cell or tissue type, such as at least one immune cell type, relative to the degree of methylation in the same region of DNA from at least one other cell or tissue type, such as other immune cell types, or from the same cell or tissue type from a healthy subject.

A nucleic acid is “produced by a tumor” if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′-3′ direction of the nucleic acid sequence)). In some embodiments, DNA methylation refers to addition of a methyl group to adenine, such as in N6-methyladenine. In some embodiments, DNA methylation is 5-methylation (modification of the carbon at position 5 of the 6-membered ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the nitrogen at position 3 of the 6-membered ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of nucleic acid molecule(s) relative to the other nucleic acid molecules comprising the same genetic information within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules comprising the same genetic information within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase.

In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. Suitable agents include agents that recognize modified nucleotides in double-stranded DNA, single-stranded DNA, and both double-stranded and single-stranded DNA.

The term “epigenetic status” refers to a certain level or extent of a sequence-independent variable that may be present in a DNA sequence. In some embodiments, the epigenetic status of a DNA sequence refers to the extent or level of methylation, nucleosome distribution, cfDNA fragmentation pattern, CCCTC-binding factor (“CTCF”) binding, transcription start site, or regulatory protein binding region of the sequence. Epigenetic statuses thus include, but are not limited to, hypermethylation, hypomethylation, and the presence of absence of CTCF binding sites or transcription start sites. The epigenetic status of a sequence may be a “reference epigenetic status” that can be used for comparison to the epigenetic status of the corresponding sequence in other DNA molecules. An example of a reference epigenetic status is a status that is prevalent in samples obtained from healthy subjects and is not associated with cancer.

As used herein, “methylation status” refers to the presence or absence of a methyl group on a DNA nucleobase (e.g. cytosine) at a particular genomic position in a nucleic acid, the degree of methylation of a nucleic acid (e.g., high, low, intermediate, or unmethylated), or the number of nucleotides methylated in a particular nucleic acid molecule. A nucleic acid “in methylated form” means that it comprises a sequence containing a methylated DNA nucleobase, e.g., a methylated cytosine in a CpG dinucleotide.

As used herein, “methylation-sensitive nuclease” refers to a nuclease that preferentially cuts unmethylated DNA relative to methylated DNA. For example, a methylation-sensitive nuclease may cut at or near a recognition sequence such as a restriction site in a manner dependent on lack of methylation of at least one of the nucleobases in the recognition sequence, such as a cytosine. In some embodiments, the nucleolytic activity of the methylation-sensitive nuclease is at least 10, 20, 50, or 100-fold higher on an unmethylated recognition site relative to a methylated control in a standard nucleolysis assay. Methylation-sensitive nucleases include methylation-sensitive restriction enzymes.

As used herein, “methylation sensitive restriction enzyme” or “MSRE” refers to a methylation sensitive nuclease that is a restriction enzyme. An MSRE is sensitive to the methylation status of the DNA (e.g. cytosine methylation), i.e., the presence or absence of methyl group in a nucleotide base in its recognition sequence alters the rate at which the enzyme cleaves the DNA. In some embodiments, the methylation sensitive restriction enzymes do not cleave the DNA if a particular nucleotide base is methylated at the recognition sequence. For example, HpaII is a methylation sensitive restriction enzyme with a recognition sequence “CCGG” and it does not cleave DNA if the second cytosine in the recognition sequence is methylated.

As used herein, “methylation-dependent nuclease” refers to a nuclease that preferentially cuts methylated DNA relative to unmethylated DNA. For example, a methylation-dependent nuclease may cut at or near a recognition sequence such as a restriction site in a manner dependent on methylation of at least one of the nucleobases in the recognition sequence, such as a cytosine. In some embodiments, the nucleolytic activity of the methylation-dependent nuclease is at least 10, 20, 50, or 100-fold higher on a methylated recognition site relative to an unmethylated control in a standard nucleolysis assay. Methylation-dependent nucleases include methylation-dependent restriction enzymes.

As used herein, “methylation-dependent restriction enzyme” or “MDRE” refers to a methylation dependent nuclease that is a restriction enzyme. An MDRE is dependent on methylation of the DNA (e.g. cytosine methylation) i.e., the presence or absence of methyl group in a nucleotide base alters the rate at which the enzyme cleaves the DNA. In some embodiments, the methylation dependent restriction enzymes do not cleave the DNA if a particular nucleotide base is unmethylated at the recognition sequence. For example, MspJI is a methylation dependent restriction enzyme with a recognition sequence “mCNNR(N9)” and it does not cleave DNA if the absence of the methylated cytosine (mC) in the recognition sequence.

As used herein, “digestion efficiency” or “cutting efficiency” refers to to the efficiency of restriction enzyme digestion. The digestion efficiency can be calculated based on the number of control molecules observed upon digesting with restriction enzyme and number of control molecules observed in the absence of restriction enzyme digestion. The MSRE digestion efficiency can be calculated by: Efficiency=1−(number of negative control molecules_{[MSRE]}/number of negative control molecules_{[Mock]}). The MDRE digestion efficiency can be calculated by: Efficiency=1−(number of positive control molecules_{[MDRE]}/number of positive control molecules_{[Mock]}).

As used herein, “mutation” refers to a variation from a known reference sequence and includes mutations such as, for example, single nucleotide variants (SNVs), and insertions or deletions (indels). A mutation can be a germline or somatic mutation. In some embodiments, a reference sequence for purposes of comparison is a wildtype genomic sequence of the species of the subject providing a test sample, typically the human genome.

As used herein, the terms “neoplasm” and “tumor” are used interchangeably. They refer to abnormal growth of cells in a subject. A neoplasm or tumor can be benign, potentially malignant, or malignant. A malignant tumor is a referred to as a cancer or a cancerous tumor.

As used herein, “nucleic acid tag” refers to a short nucleic acid (e.g., less than about 500 nucleotides, about 100 nucleotides, about 50 nucleotides, or about 10 nucleotides in length), used to distinguish nucleic acids from different samples (e.g., representing a sample index), distinguish nucleic acids from different partitions (e.g., representing a partition tag) or different nucleic acid molecules in the same sample (e.g., representing a molecular barcode), of different types, or which have undergone different processing. The nucleic acid tag comprises a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples comprising nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g. molecular identifier, sample identifier). Additionally, or alternatively, nucleic acid tags can be used as molecular identifiers (e.g., to distinguish between different molecules or amplicons of different parent molecules in the same sample or sub-sample). This includes, for example, uniquely tagging different nucleic acid molecules in a given sample, or non-uniquely tagging such molecules. In the case of non-unique tagging applications, a limited number of tags (i.e., molecular barcodes) may be used to tag each nucleic acid molecule such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

As used herein, “partitioning” refers to physically separating, sorting, and/or fractionating a mixture of nucleic acid molecules in a sample into a plurality of subsamples or subpopulations of nucleic acids based on a characteristic of the nucleic acid molecules. A sample or population may be partitioned into one or more partitioned subsamples or subpopulations based on a characteristic that is indicative of a genetic or epigenetic change or a disease state. The partitioning can be physical partitioning of molecules. Partitioning can involve separating the nucleic acid molecules into groups or sets based on the level of epigenetic feature (for e.g., methylation). For example, the nucleic acid molecules can be partitioned based on the level of methylation of the nucleic acid molecules. Stated differently, partitioning may include physically partitioning nucleic acid molecules based on the presence or absence of one or more methylated nucleobases. In some embodiments, the methods and systems used for partitioning may be found in PCT Patent Application No. PCT/US2017/068329, which is hereby incorporated by reference in its entirety.

As used herein, “partitioned set” or “partition” refers to a set of nucleic acid molecules partitioned into a set or group based on the differential binding affinity of the nucleic acid molecules or proteins associated with the nucleic acid molecules to a binding agent. A partitioned set may also be referred to as a subsample. The binding agent binds preferentially to the nucleic acid molecules comprising nucleotides with epigenetic modification. For example, if the epigenetic modification is methylation, the binding agent can be a methyl binding domain (MBD) protein. In some embodiments, a partitioned set can comprise nucleic acid molecules belonging to a particular level or degree of epigenetic feature (for e.g., methylation). For example, the nucleic acid molecules can be partitioned into three sets—one set for highly methylated nucleic acid molecules (first subsample, hyper partition, hyper partitioned set or hypermethylated partitioned set), a second set for low methylated nucleic acid molecules (second subsample, hypo partition, hypo partitioned set or hypomethylated partitioned set), and a third set for intermediate methylated nucleic acid molecules (third subsample, intermediate partitioned set, intermediately methylated partitioned set, residual partition, or residual partitioned set). In another example, the nucleic acid molecules can be partitioned based on the number of methylated nucleotides—one partitioned set can have nucleic acid molecules with nine methylated nucleotides, and another partitioned set can have unmethylated nucleic acid molecules (zero methylated nucleotides).

As used herein, “sample” means anything capable of being analyzed by the methods and/or systems disclosed herein.

As used herein, “sequencing” refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, long-read sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina, Inc., Pacific Biosciences, Inc., or Applied Biosystems/Thermo Fisher Scientific, among many others.

As used herein, “long-read sequencing” refers to sequencing methods that can generate longer sequencing reads, such as reads in excess of 10 kilobases, as compared to short-read sequencing methods (such as Illumina NovaSeq, HiSeq, NextSeq, and MiSeq instruments, BGI MGISEQ and BGISEQ models, or Thermo Fisher Ion Torrent sequencers), which generally produce reads of up to about 600 bases in length. Long-read sequencing methods may be referred to as “third generation sequencing.” Compared to short reads, long reads can improve de novo assembly, transcript isoform identification, and detection of structural variants mapping certainty. Furthermore, long-read sequencing of native DNA or RNA molecules reduces amplification bias and preserves base modifications. Long-read sequencing technologies useful herein can include any suitable long-read sequencing methods, such as, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping.

As used herein, “nanopore sequencing” refers to sequencing methods that directly sequence a single-stranded DNA molecule (such as a native DNA molecule) by measuring characteristic signals (e.g., current changes) as the bases are threaded through a nanopore of a membrane, such as by a molecular motor protein. As such, nanopore sequencing methods can produce reads that are significantly longer than those produced by next-generation (also known as second generation) sequencing technologies.

As used herein, “5-letter sequencing” and “6-letter sequencing” refers to sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout, respectively, in a single workflow. Such methods may use enzymatic processing of a DNA sample, rather than, e.g., bisulfite treatment.

As used herein, “next-generation sequencing” or “NGS” refers to sequencing technologies having increased throughput as compared to traditional Sanger- and capillary electrophoresis-based approaches, for example, with the ability to generate hundreds of thousands of relatively small sequence reads at a time. Some examples of next-generation sequencing techniques include, but are not limited to, sequencing by synthesis, sequencing by ligation, and sequencing by hybridization. In some embodiments, next-generation sequencing includes the use of instruments capable of sequencing single molecules. Example of commercially available instruments for performing next-generation sequencing include, but are not limited to, NextSeq, HiSeq, NovaSeq, MiSeq, Ion PGM and Ion GeneStudio S5.

As used herein, the terms “somatic mutation” or “somatic variation” are used interchangeably. They refer to a mutation in the genome that occurs after conception. Somatic mutations can occur in any cell of the body except germ cells and accordingly, are not passed on to progeny.

As used herein, “subject” refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms “individual” or “patient” are intended to be interchangeable with “subject”. For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

“Or” is used in the inclusive sense, i.e., equivalent to “and/or,” unless the context requires otherwise.

### II. Exemplary Methods

### A. Overview

Cancer formation and progression may arise from both genetic modification and epigenetic features of deoxyribonucleic acid (DNA). The present disclosure provides methods and systems for analyzing DNA, such as cell-free DNA (cfDNA). The present disclosure provides single-base resolution sequencing methods that incorporate barcoding and methylation-specific amplification, and exhibit increased sensitivity (e.g., ability to detect epigenetic features reliably in samples containing fewer molecules) and/or reduced molecular losses compared to other sequencing methods that include a pre-amplification epigenetic base conversion step.

Without wishing to be bound by any particular theory, cells in or around a cancer or neoplasm may shed more DNA than cells of the same tissue type in a healthy subject. As such, the distribution of tissue of origin of certain DNA samples, such as cfDNA, may change upon carcinogenesis. Thus, for example, an increase in the level of hypermethylation variable target regions that show lower methylation in healthy cfDNA than in at least one other tissue type can be an indicator of the presence (or recurrence, depending on the history of the subject) of cancer. Similarly, an increase in the level of hypomethylation variable target regions in the sample can be an indicator of the presence (or recurrence, depending on the history of the subject) of cancer.

Additionally, cancer can be indicated by non-sequence modifications, such as methylation. Examples of methylation changes in cancer include local gains of DNA methylation in the CpG islands at the TSS of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression.

Thus, DNA methylation profiling can be used to detect aberrant methylation in DNA of a sample. The DNA can correspond to certain genomic regions (“differentially methylated regions” or “DMRs”) that are normally hypermethylated or hypomethylated in a given sample type (e.g., cfDNA from the bloodstream) but which may show an abnormal degree of methylation that correlates to a neoplasm or cancer, e.g., because of unusually increased contributions of tissues to the type of sample (e.g., due to increased shedding of DNA in or around the neoplasm or cancer) and/or from extents of methylation of the genome that are altered during development or that are perturbed by disease, for example, cancer or any cancer-associated disease.

In some embodiments, DNA methylation comprises addition of a methyl group to a cytosine residue at a CpG site (cytosine-phosphate-guanine site (i.e., a cytosine followed by a guanine in a 5′->3′ direction of the nucleic acid sequence). In some embodiments, DNA methylation comprises addition of a methyl group to an adenine residue, such as in N6-methyladenine. In some embodiments, DNA methylation is 5-methylation (modification of the carbon at position 5 of the 6-membered ring of cytosine). In some embodiments, 5-methylation comprises addition of a methyl group to the 5C position of the cytosine residue to create 5-methylcytosine (m5c or 5-mC or 5mC). In some embodiments, methylation comprises a derivative of m5c. Derivatives of m5c include, but are not limited to, 5-hydroxymethylcytosine (5-hmC or 5hmC), 5-formylcytosine (5-fC), and 5-caryboxylcytosine (5-caC). In some embodiments, DNA methylation is 3C methylation (modification of the nitrogen at position 3 of the 6-membered ring of the cytosine residue). In some embodiments, 3C methylation comprises addition of a methyl group to the 3C position of the cytosine residue to generate 3-methylcytosine (3mC). Methylation can also occur at non-CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

Methylation profiling can involve determining methylation patterns across different regions of the genome. This can show regions of the genome that, compared with other regions, are more highly methylated or are less highly methylated. In this way, genomic regions, in contrast to individual molecules, may differ in their extent of methylation.

However, current single-base resolution sequencing methods may lead to loss of input DNA, e.g., recovering only about 1-25% of input DNA molecules. These base conversion steps generally are performed before amplification. To overcome this issue, methods attempting to amplify DNA while maintaining the original DNA methylation patterns have been developed. These methods involve use of a methyltransferase, DNMT1, that in nature maintains base methylation status during DNA replication, preferentially methylating the opposite strand of (CpG) of hemi-methylated substrates (5mCpG). In practice, however, DNMT1 exhibits imperfect preservation of methylation in in vitro amplification, in that there is generally <100% efficiency at methylating across hemi-methylated sites and/or >0% efficiency at methylating non-methylated dsDNA sites (i.e., de novo methylation). This limits the utility of current methylation-preserving amplification techniques. While molecular losses can be reduced compared to standard methylation sequencing (or qPCR) methods, accuracy may be limited.

In an aspect, the present disclosure provides a solution for overcoming the current limitations of methylation-preserving amplification by using molecular barcodes in methylation-preserving amplification, e.g., to correct or eliminate errors in methyltransferase activity. In other embodiments, barcodes are not employed, and the methylation-preserving amplification is a linear amplification method, wherein errors generated by the methyltransferase are not propagated exponentially in the resultant family of DNA copies. Without being bound to a particular theory, an original template DNA molecule comprises the “correct” epigenetic marks (i.e., the epigenetic marks of the sample DNA, prior to introduction of any errors during sample processing, such as during an amplification step). Thus, in some embodiments wherein the DNA is linearly amplified using a methylation-preserving amplification method, DNA copies are synthesized only from the original template molecules (e.g., using only a reverse primer), reducing error as compared to standard exponential amplifications (such as PCR with forward and reverse primers, which amplifies original and copy strands, thereby potentially amplifying strands comprising errors introducing during the amplification step).

Methods described herein in which barcodes are used result in a family of DNA copies generated from an original template with shared molecular barcode(s) that can be identified during sequencing. Sequence errors from DNA polymerase, and methylation errors from DNMT1 infidelity are seen as variation between DNA copies from the same family (e.g., DNA molecules), wherein DNA copies generated from an original template molecule have (a) a shared molecular barcode, (b) the same genomic start position, (c) the same genomic stop position, or a combination of (a) with (b) and/or (c), at a given base. After epigenetic base conversion, an epigenetic consensus sequence can be determined from a plurality of reads of a family, such as from all the copies/NGS reads of a family, thereby suppressing errors generated during the methylation-preserving amplification.

It is also possible to correct sequence errors introduced in vitro by DNA polymerase in this workflow. Base conversion workflows that convert methylated bases (such as TAPS, which converts methylated cytosine to thymine) may be used in conjunction with the disclosed methylation-preserving amplification methods, to suppress both genetic and epigenetic errors.

In some embodiments, a method of analyzing DNA comprises, in the following order (as indicated by lettering), (a) performing a methylation-preserving amplification of the DNA, wherein the DNA comprises barcodes; and (b) sequencing the DNA in a modification-sensitive manner and determining an epigenetic consensus sequence of the DNA associated with at least a portion of the barcodes. In some embodiments, a method of analyzing DNA comprises, in the following order (as indicated by lettering), (a) performing a methylation-preserving amplification of the DNA, wherein the DNA comprises barcodes; (b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and (c) sequencing the DNA and determining an epigenetic consensus sequence of the DNA associated with at least a portion of the barcodes. In some embodiments, a method of analyzing DNA comprises, in the following order (as indicated by lettering), (a) performing a methylation-preserving amplification of the DNA, wherein the DNA comprises barcodes; (b) enriching for one or more sets of epigenetic target regions of DNA from the DNA, thereby providing enriched DNA; (c) sequencing the enriched DNA in a modification-sensitive manner and determining an epigenetic consensus sequence of the enriched DNA associated with at least a portion of the barcodes. In some embodiments, a method of analyzing DNA comprises, in the following order (as indicated by lettering), (a) performing a linear, methylation-preserving amplification of the DNA; and (b) sequencing the DNA in a modification-sensitive manner and determining an epigenetic consensus sequence of the DNA. In some embodiments, a method of analyzing DNA comprises, in the following order (as indicated by lettering), (a) performing a linear, methylation-preserving amplification of the DNA; (b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and (c) sequencing the DNA and determining an epigenetic consensus sequence of the DNA. In some embodiments, a method of analyzing DNA comprises, in the following order (as indicated by lettering), (a) performing a linear, methylation-preserving amplification of the DNA; (b) enriching for one or more sets of epigenetic target regions of DNA from the DNA, thereby providing enriched DNA; (c) sequencing the enriched DNA in a modification-sensitive manner and determining an epigenetic consensus sequence of the enriched DNA.

In certain embodiments wherein a conversion method affects unmethylated cytosines (e.g., bisulfite conversion, EM-Seq, or SEM-Seq, as described elsewhere herein), if an adapter ligated to the DNA comprises cytosines in non-CpG contexts, (1) if the cytosines are methylated, this methylation may not be propagated by a DNA methyltransferase (e.g., DNMT1), and (2) if the cytosines are unmethylated (which they generally will be after amplification), they will be deaminated during base conversion using a reagent that acts on unmethylated cytosines, such as a deaminase enzyme or bisulfite. To address these limitations, in some embodiments of the disclosed methods, a methylation-preserving amplification of the DNA is performed, wherein an adapter attached to the DNA comprises a barcode and a restriction enzyme cleavage site between the barcode and at least a portion of the adapter. In such embodiments, the barcode is located such that cleavage of the adapter at the restriction enzyme cleavage site by a restriction enzyme does not remove the barcode from the DNA, e.g., the barcode is located between the DNA sequence to be analyzed and the restriction site. For example, in an adapter ligated to the 5′ end of a DNA sequence to be analyzed, the barcode is 3′ of the restriction site and 5′ of the DNA sequence to be analyzed. Thus, in some embodiments of the disclosed methods, a methylation-preserving amplification of the DNA is performed, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site, thereby providing amplified DNA. In such embodiments, an insert can comprise DNA to be analyzed, such as DNA from a sample (e.g., cfDNA or DNA from a sample (such as a sample from a subject) comprising cells), or from an amplicon thereof. Further, in some embodiments, the barcodes do not comprise cytosines in a non-CpG context. For example, in some embodiments, wherein the procedure that affects a first nucleobase in the DNA differently from a second nucleobase in the DNA of the first subsample comprises enzymatic-methyl (EM) base conversion (EM-Seq) (or another base conversion method that converts unmethylated cytosines, such as bisulfite conversion or SEM-Seq as described elsewhere herein), the barcodes may not comprise cytosines in a non-CpG context. In such embodiments, if barcodes do comprise cytosines in a non-CpG context, these cytosines will be converted to uracils (and read as thymines during sequencing), and accordingly, design and sequencing of such barcodes should consider this to avoid base miscalling in the barcodes during sequencing. In other embodiments, wherein the procedure that affects a first nucleobase in the DNA differently from a second nucleobase in the DNA of the first subsample comprises Tet-assisted pyridine borane sequencing (TAPS) (or another base conversion method that does not convert unmethylated cytosines), the barcodes may or may not comprise cytosines in a non-CpG context.

In some embodiments, such as embodiments wherein a methylation preserving amplification step comprises rolling circle amplification, a DNA molecule to be analyzed is circular. In such embodiments, “a restriction enzyme cleavage site between a barcode and a portion of the adapter” means that the restriction enzyme cleavage site is located in the shortest path along the molecule from the barcode to the portion of the adapter.

In some embodiments, the DNA is then contacted with a restriction enzyme (e.g., a restriction endonuclease) that cleaves the DNA at the restriction enzyme cleavage site in the adapter. In some embodiments, cleavage of the DNA by the restriction enzyme results in an overhang (such as an A or T overhang), e.g., that can be used as a sticky end for a later ligation. Before or after the contacting step (such as after the contacting step), the DNA is subjected to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some such embodiments, the first nucleobase is an unmethylated cytosine and the subjecting step (e.g., bisulfite conversion, EM-Seq, or SEM-Seq, as described elsewhere herein) affects the unmethylated cytosine. Supplemental adapters (which in some embodiments do not comprise barcodes) are then ligated to the DNA, and a uracil- and/or dihydrouracil-tolerant amplification of the DNA is performed, such as PCR using a uracil- and/or dihydrouracil tolerant DNA polymerase (e.g., Q5U® Hot Start High-Fidelity DNA Polymerase from New England Biolabs, VeraSeq™ ULtra DNA Polymerase from Qiagen, or Phusion™ U Hot Start DNA Polymerase from Thermo Fisher). In some embodiments, the DNA is then enriched for one or more sets of epigenetic target regions and/or sequence-variable target regions of DNA, thereby providing enriched DNA. In some embodiments, the enriched DNA is amplified, wherein the amplification comprises differentially tagging DNA enriched for, e.g., one or more epigenetic target region sets and/or for one or more sequence variable target region sets. In some such embodiments, the differential tagging comprises attaching sample indices to the DNA, such as by ligation or by incorporation using PCR primers.

In particular embodiments, the step of contacting the DNA with a restriction enzyme that recognizes and cleaves the DNA at the restriction enzyme cleavage site in the adapter is performed prior to the step of subjecting the DNA to a procedure that affects a first nucleobase in the DNA differently from a second nucleobase in the DNA; the contacting step may also or alternatively be performed prior to or after an enriching step. However, in embodiments wherein the restriction enzyme cleavage site does not comprise cytosines in a non-CpG context, the step of contacting the DNA with a restriction enzyme that recognizes and cleaves the DNA at the restriction enzyme cleavage site in the adapter may be performed (a) prior to or after the step of subjecting the DNA to a procedure that affects a first nucleobase in the DNA differently from a second nucleobase in the DNA, (b) prior to or after an enriching step, and (c) prior to the optional step of amplifying the enriched DNA, wherein the amplifying comprises differentially tagging DNA enriched for, e.g., one or more epigenetic target region sets and/or for one or more sequence variable target region sets.

The DNA is then sequenced, and an epigenetic consensus sequence of the DNA associated with at least a portion of the barcodes is determined. In some embodiments, an epigenetic consensus sequence of the DNA associated with at least a portion of the barcodes is determined by performing bioinformatics analysis of the sequencing (e.g., NGS) data. In such embodiments, one or more components (e.g., a barcode or set of barcodes in combination with a genomic start site and/or a genomic stop site) of each read is used to identify and group reads into molecular families (e.g., DNA molecules), wherein the reads within a molecular family have (a) a common molecular barcode (i.e., a set of reads wherein each read comprises a barcode having the same sequence) and (b) the same genomic start position, (c) having the same genomic stop position, or (d) both (b) and (c).

Restriction enzymes can recognize and bind to specific nucleotide sequences of DNA (“restriction sites,” “restriction enzyme cleavage sites”), subsequently cleaving the DNA into fragments via hydrolysis of the phosphodiester backbone at a location within the restriction site. Restriction enzymes useful for cleaving a restriction enzyme cleavage site in an adapter disclosed herein are known in the art, and may produce either blunt or sticky ends following cleavage. Sticky ends resulting from cleavage typically have 3′- or 5′-overhangs of one to four (or more) nucleotides. In some embodiments, a restriction enzyme is used that produces a single base overhang, such as a single base 5′-overhang. In some embodiments, the single base overhang (such as a single base 5′-overhang) is a T. In some embodiments, the single base overhang (such as a single base 5′-overhang) is an A. In embodiments wherein, following a conversion step and optionally an enrichment step, a supplemental adapter is ligated to the DNA to be analyzed, sticky end or blunt end ligation may be used. In a particular embodiment, a restriction enzyme used to cleave an adapter at a restriction enzyme cleavage site produces sticky ends, and a supplemental adapter (such as an adapter that does not comprise a barcode) is ligated to the DNA using sticky end ligation following an enrichment step, and prior to an optional step of amplifying the enriched DNA.

In some embodiments, a method of analyzing DNA comprises, in the following order (as indicated by lettering), (a) performing a methylation-preserving amplification of the DNA, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site, thereby providing amplified DNA; (b) contacting the amplified DNA with a restriction enzyme that recognizes and cleaves the DNA at the restriction enzyme cleavage site in the adapter; (c) before or after step (b), subjecting the amplified DNA to a procedure that affects a first nucleobase of the amplified DNA differently from a second nucleobase of the amplified DNA, wherein the first nucleobase is a modified or unmodified nucleobase, the second nucleobase is a modified or unmodified nucleobase different from the first nucleobase, and the first nucleobase and the second nucleobase have the same base pairing specificity; (d) after steps (b) and (c), ligating supplemental adapters to the amplified DNA; (e) after step (d), performing a uracil- and/or dihydrouracil-tolerant amplification of the DNA; (f) after step (e), enriching for one or more sets of epigenetic target regions of DNA from the amplified DNA, thereby providing enriched DNA; (g) optionally further amplifying the enriched DNA; and (h) sequencing the enriched DNA and determining an epigenetic consensus sequence of the DNA associated with at least a portion of the barcodes. In some embodiments, the barcodes do not comprise cytosines in a non-CpG context. In some embodiments, adapters ligated to the DNA in step (d) do not comprise barcodes. In some embodiments, the optional step of amplifying the enriched DNA is performed and further comprises differentially tagging the enriched DNA. In some such embodiments, differentially tagging the enriched DNA comprises attaching one or more sample indices to the DNA, such as by ligation or by incorporation using PCR primers. In some embodiments, cleavage of the DNA by the restriction enzyme results in an overhang, such as a single base overhang, such as a single base 5′-overhang. In some such embodiments, the single base overhang is a T. In other such embodiments, the single base overhang is an A.

### B. Amplification

In some embodiments, DNA is amplified. For example, DNA flanked by adapters added to the DNA as described herein can be amplified using a methylation-preserving amplification method. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). In some embodiments, DNA is amplified using linear amplification with thermocycling and DNMT1 (Chang, et al., “DNA 5-Methylcytosine-Specific Amplification and Sequencing,” J. Am. Chem. Soc. 2020, 142(10):4539-4543). Other amplification methods of use herein include the ligase chain reaction, strand displacement amplification, nucleic acid sequence-based amplification, and self-sustained sequence-based replication. In some embodiments, detecting the presence or absence of one or more DNA sequences comprises a methylation-preserving amplification, such as an amplification performed in the presence of a methyltransferase.

Methylating agents of use in methylation-preserving amplification methods described herein are known to those of ordinary skill in the art, and can include, for example, any suitable methyltransferase. In some embodiments, the methylating agent is DNMT1. DNMT1 is the most abundant DNA methyltransferase in mammalian cells and predominantly methylates hemimethylated CpG di-nucleotides in the mammalian genome. For example, DNA molecules replicated using PCR amplification with DNMT1 incubation will maintain their methylation status post-amplification, for use in further analyses, such as those described herein (such as an epigenetic base conversion step and/or an enrichment step).

Additional methylating agents useful herein include the mammalian methyltransferases, DNMT3a and DNMT3b, the plant methyltransferases, MET1, and CMT3. In some embodiments, DNMT1 or another suitable methyltransferase is used with a methyl donor and may be used with or without cofactors known to those of ordinary skill in the art. DNMT1 works in vitro at 95% efficiency without a cofactor; however, DNMT1 may be used with a cofactor such as NP95(Uhrfl), such as described in Bashtrykov P I, et al. “The UHRF1 protein stimulates the activity and specificity of the maintenance DNA methyltransferase DNMT1 by an allosteric mechanism.” J Biol Chem. 2014. In some embodiments, DNMT1 is used at a concentration of about 50-10000 U/mL, such as about 50-2000, about 50-5000, about 2500-7500, or about 5000-10000 U/mL. In some embodiments, DNMT1 is used at a concentration of about 100-500, about 500-1000, about 100-1000, about 1000-1500, about 500-1500, about 600-1400, about 700-1300, about 800-1200, about 900-1100, or about 950-1050 U/mL. In some embodiments, DNMT1 is used at a concentration of about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 U/mL. In some embodiments, DNMT1 is used at a concentration of about 1,000 U/ml.

Some PCR enzymes, such as archaeal polymerases, do not efficiently copy, e.g., bisulfite-treated DNA due to stalling triggered by template uracil. Thus, in embodiments wherein DNA is amplified following a conversion step that produces uracils or dihydroxyuracils (e.g., bisulfite conversion, SEM-Seq, or TAPs), the amplification may comprise a uracil- and/or dihydrouracil-tolerant amplification, such as PCR using a uracil- and/or dihydrouracil-tolerant DNA polymerase. Exemplary such polymerases are known in the art and are commercially available (e.g., Q5U® Hot Start High-Fidelity DNA Polymerase from New England Biolabs, VeraSeq™ ULtra DNA Polymerase from Qiagen, and Phusion™ U Hot Start DNA Polymerase from Thermo Fisher).

### C. Adapter Ligation or Addition; Tagging

In some embodiments, the disclosed methods comprise adding adapters to DNA. In some embodiments, adapters are added (e.g., “attached”) to the DNA before or during an amplification step, such as a methylation-preserving amplification step, before or after subjecting the DNA to a procedure that affects a first nucleobase in the DNA differently from a second nucleobase in the DNA, and/or before or after enrichment of epigenetic and/or sequence-variable target regions of the DNA. In some embodiments, adapters may be added to DNA concurrently with an amplification procedure (such as a methylation-preserving amplification step), e.g., by providing the adapters in a 5′ portion of a primer (where PCR is used, this can be referred to as library prep-PCR or LP-PCR), before or after an amplification step. In some embodiments, adapters are added by other approaches. In some such methods, first adapters are added to the nucleic acids by ligation to the 3′ ends thereof, which may include ligation to single-stranded DNA. In other such methods, first adapters are added to the nucleic acids by ligation to the 5′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified. In some embodiments, end repair of the DNA is performed prior to addition of adapters.

In a particular embodiment, first adapters are added to the nucleic acids by ligation to the 3′ ends thereof, which may include ligation to single-stranded DNA. A methyl-preserving linear amplification is then performed using the 3′ adapter as a primer binding site, followed by a step of subjecting the DNA to a procedure that affects a first nucleobase of the DNA differently from a second nucleobase of the DNA, wherein the first nucleobase is an unmethylated cytosine and the subjecting step (e.g., bisulfite conversion, EM-Seq, or SEM-Seq, as described elsewhere herein) affects the unmethylated cytosine. The amplified, converted DNA is then subjected to a single-stranded DNA library preparation step.

In some embodiments, the single-stranded DNA library preparation is performed in a one-step combined phosphorylation/ligation reaction, e.g., as described in Troll et al., BMC Genomics, 20:1023 (2019), DOI: 10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, following attachment of adapters, the nucleic acids are subject to amplification, such as methylation-preserving amplification. The amplification can use, e.g., universal primers that recognize primer binding sites in the adapters.

In some embodiments, the DNA is linked at both ends to Y-shaped adapters including primer binding sites and tags. In some such embodiments, the DNA is amplified.

Tagging DNA molecules is a procedure in which a tag is attached to or associated with the DNA molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another in both unique and non-unique tagging scenarios). For methods that involve a partitioning step, a partition tag (which distinguishes molecules in one partition from those in a different partition) may be included. In some embodiments, adapters added to DNA molecules comprise tags. In some such embodiments, the tag comprises one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. In some embodiments, the non-unique tags comprise non-unique barcodes. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites. In some embodiments, adapters include the same primer binding site.

In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. In some embodiments comprising barcode attachment, e.g., randomly, to both ends of a molecule, the combination of barcodes, together, constitutes a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z 6*z, 7*z 8*z, 9*z, 10*z, 11*z 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags (e.g., non-unique barcodes) in reactions follows methods and systems described by US patent applications 20010053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments, 20-50 different tags (e.g., barcodes) are ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, two or more populations, samples, subsamples, or partitions are differentially tagged, such as partitioned subsamples and/or subsamples that are differentially degraded using one or more methylation-sensitive nucleases. Tags can be used to label the individual DNA populations so as to correlate the tag (or tags) with a specific population or partition. In some embodiments, a single tag can be used to label a specific population or partition. In some embodiments, multiple different tags can be used to label a specific population or partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108: 9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after tagging, the molecules may be pooled for sequencing in a single run. In some embodiments, a sample tag is added to the molecules, e.g., in a step subsequent to addition of other tags and pooling. Sample tags can facilitate pooling material generated from multiple samples for sequencing in a single sequencing run.

In some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules based on one or more characteristics, the final tagged molecules in the library may no longer possess that characteristic. For example, while single stranded DNA molecules may be partitioned and/or tagged, the final tagged molecules in the library are likely to be double stranded. Similarly, while DNA may be subject to partition based on different levels of methylation, in the final library, tagged molecules derived from these molecules are likely to be unmethylated (such as following a conversion step and/or further amplification step as disclosed herein). Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

In some embodiments, an adapter of use herein comprises a barcode and a restriction enzyme cleavage site. In some embodiments, DNA to be analyzed in a disclosed method comprises inserts and adapters comprising barcodes, at least one of the adapters further comprises a restriction enzyme cleavage site between a barcode and a portion of the adapter, and the barcode is located between the insert and the restriction enzyme cleavage site. In such embodiments, cleavage of the adapter at the restriction enzyme cleavage site by a restriction enzyme does not remove the barcode from a DNA molecule to which the adapter has been ligated. In some embodiments, the barcode does not comprise cytosines in a non-CpG context.

After sequencing, analysis of reads can be performed on a partition-by-partition level, as well as a pooled DNA level. Tags are used to sort reads from different partitions. Analysis can include in silico analysis to determine genetic and epigenetic variation (one or more of methylation, chromatin structure, etc.) using sequence information, genomic coordinates length, coverage, and/or copy number.

D. Subjecting the DNA or a Subsample Thereof to a Procedure that Affects a First Nucleobase in the DNA Differently from a Second Nucleobase in the DNA

In some embodiments, methods disclosed herein comprise a step of subjecting DNA, or a subsample thereof, to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, DNA is subjected to a procedure that affects a first nucleobase in the DNA differently from a second nucleobase in the DNA after methylation-preserving amplification, and before sequencing. In certain embodiments, the DNA is subjected to the procedure before the DNA is enriched for one or more epigenetic and/or sequence-variable target regions, and/or before or after contacting the DNA with a methylation-sensitive nuclease.

In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, such as where one of the first and second nucleobases comprises mC and the other comprises hmC.

In some embodiments, the procedure that affects a first nucleobase in the DNA differently from a second nucleobase in the DNA of the first subsample comprises bisulfite conversion. Treatment with bisulfite converts unmodified cytosine and certain modified cytosine nucleotides (e.g., 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase comprises one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion, such as on a DNA sample as described herein, thus facilitates identifying positions containing mC or hmC using the sequence reads obtained from the exemplary sample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA of the first subsample comprises oxidative bisulfite (Ox-BS) conversion. This procedure first converts hmC to fC, which is bisulfite susceptible, followed by bisulfite conversion. Thus, when oxidative bisulfite conversion is used, the first nucleobase comprises one or more of unmodified cytosine, fC, caC, hmC, or other cytosine forms affected by bisulfite, and the second nucleobase comprises mC. Sequencing of Ox-BS converted DNA identifies positions that are read as cytosine as being mC positions. Meanwhile, positions that are read as T are identified as being T, hmC, or a bisulfite-susceptible form of C, such as unmodified cytosine, fC, or hmC. Performing Ox-BS conversion, such as on a DNA sample as described herein, thus facilitates identifying positions containing mC using the sequence reads obtained from the sample. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA of the first subsample comprises Tet-assisted bisulfite (TAB) conversion. In TAB conversion, hmC is protected from conversion and mC is oxidized in advance of bisulfite treatment, so that positions originally occupied by mC are converted to U while positions originally occupied by hmC remain as a protected form of cytosine. For example, as described in Yu et al., Cell 2012; 149: 1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Alternatively, a carbamoyltransferase enzyme, such as 5-hydroxymethylcytosine carbamoyltransferase as described in Yang et al., Bio-protocol, 2023; 12(17): e4496, can be used to protect hmC (by converting hmC to 5-carbamoyloxymethylcytosine (5cmC)), then a TET protein such as mTet1 can be used to convert mC to caC, and then bisulfite treatment can be used to convert C and caC to U while 5cmC remains unaffected. Thus, when TAB conversion is used, the first nucleobase comprises one or more of unmodified cytosine, fC, caC, mC, or other cytosine forms affected by bisulfite, and the second nucleobase comprises hmC. Sequencing of TAB-converted DNA identifies positions that are read as cytosine as being hmC positions. Meanwhile, positions that are read as T are identified as being T, mC, or a bisulfite-susceptible form of C, such as unmodified cytosine, fC, or caC. Performing TAB conversion, such as on a DNA sample as described herein, thus facilitates identifying positions containing hmC using the sequence reads obtained from the sample.

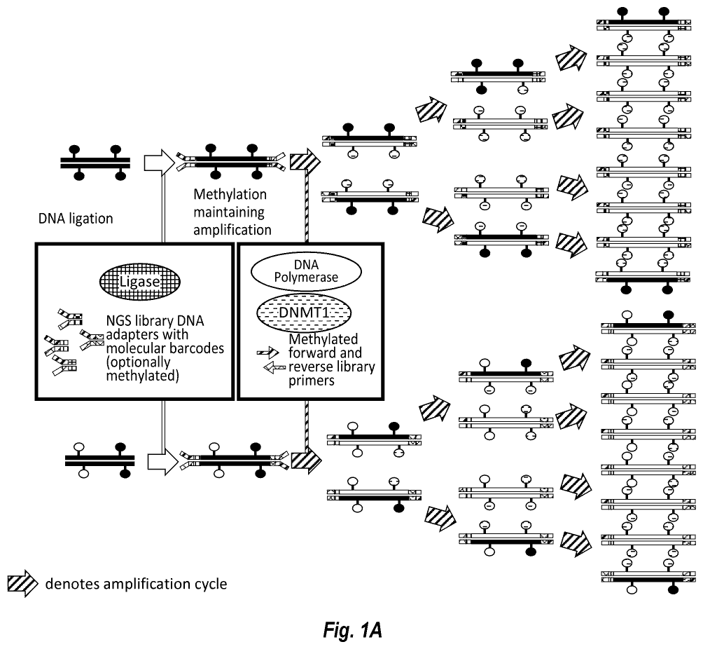

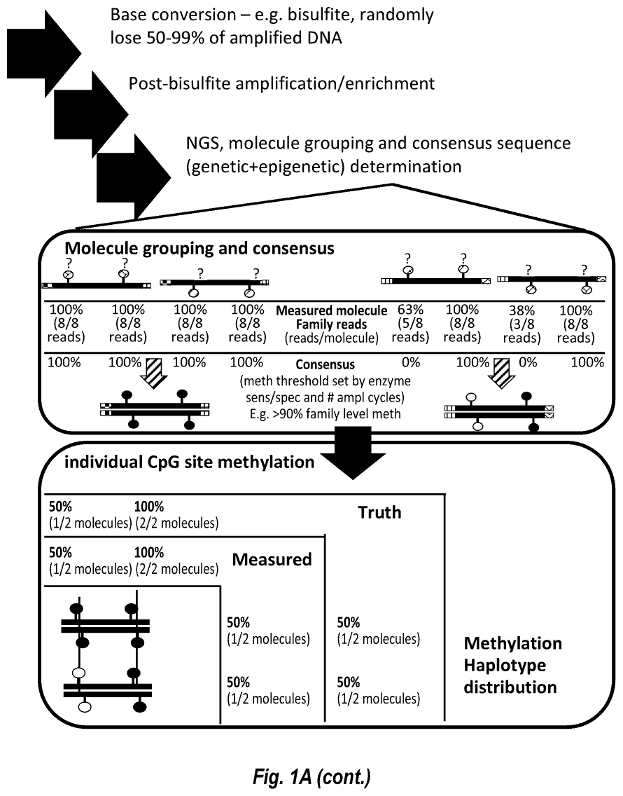

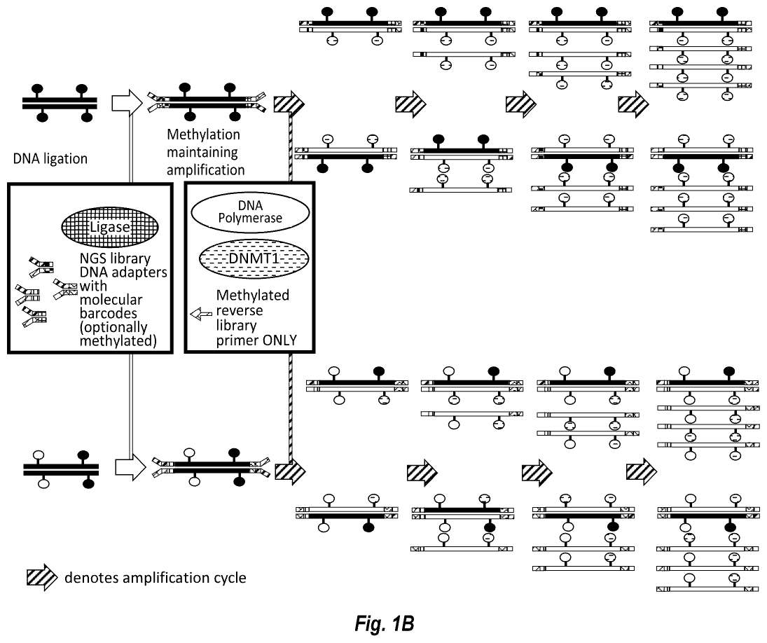

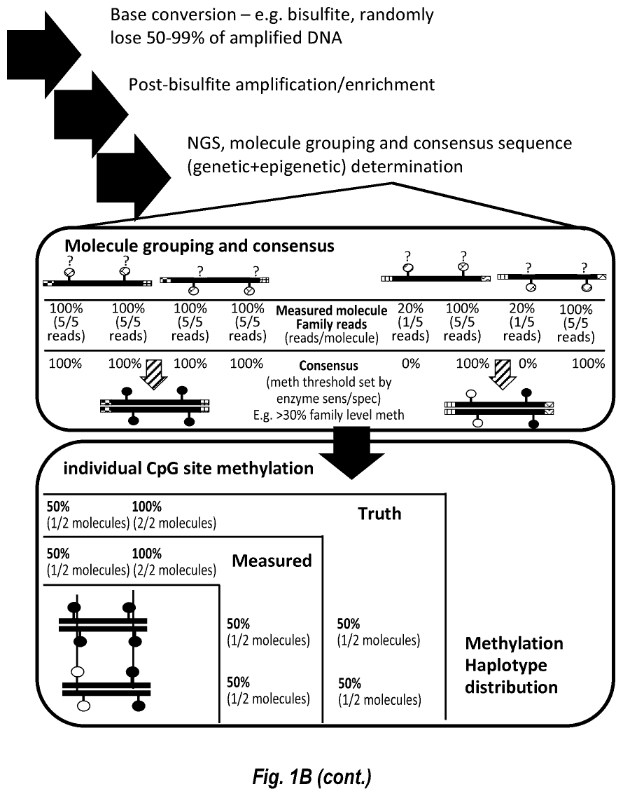

In some embodiments, the procedure that affects a first nucleobase in the DNA differently from a second nucleobase in the DNA of the first subsample comprises Tet-assisted conversion with a substituted borane reducing agent, optionally wherein the substituted borane reducing agent is 2-picoline borane, borane pyridine, tert-butylamine borane, or ammonia borane. In Tet-assisted pic-borane conversion with a substituted borane reducing agent conversion, a TET protein is used to convert mC and hmC to caC, without affecting unmodified C. caC, and fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). DHU is read as a T in sequencing. Thus, when this type of conversion is used, the first nucleobase comprises one or more of mC, fC, caC, or hmC, and the second nucleobase comprises unmodified cytosine. Sequencing of the converted DNA identifies positions that are read as cytosine as being unmodified C positions. Meanwhile, positions that are read as T are identified as being T, mC, fC, caC, or hmC. Performing TAP conversion, such as on a DNA sample as described herein, thus facilitates identifying positions containing unmodified C using the sequence reads obtained from the sample. This procedure encompasses Tet-assisted pyridine borane sequencing (TAPS), described in further detail in Liu et al. 2019, supra.

Alternatively, protection of hmC (e.g., using βGT or 5-hydroxymethylcytosine carbamoyltransferase) can be combined with Tet-assisted conversion with a substituted borane reducing agent. hmC can be protected as noted above through glucosylation using βGT, forming ghmC, or through carbamoylation using 5-hydroxymethylcytosine carbamoyltransferase, forming 5cmC. Treatment with a TET protein such as mTet1 then converts mC to caC but does not convert C, ghmC, or 5cmC. caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either hmC or unmodified C positions. Meanwhile, positions that are read as T are identified as being T, fC, caC, or mC. Performing TAPSβ conversion, such as on a DNA sample as described herein, thus facilitates distinguishing positions containing unmodified C or hmC on the one hand from positions containing mC using the sequence reads obtained from the sample. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12(17): e4496.

In some embodiments, the procedure that affects a first nucleobase in the DNA differently from a second nucleobase in the DNA of the first subsample comprises chemical-assisted conversion with a substituted borane reducing agent, optionally wherein the substituted borane reducing agent is 2-picoline borane, borane pyridine, tert-butylamine borane, or ammonia borane. In chemical-assisted conversion with a substituted borane reducing agent, an oxidizing agent such as potassium perruthenate (KRuO₄) (also suitable for use in ox-BS conversion) is used to specifically oxidize hmC to fC. Treatment with pic-borane or another substituted borane reducing agent such as borane pyridine, tert-butylamine borane, or ammonia borane converts fC and caC to DHU but does not affect mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either mC or unmodified C positions. Meanwhile, positions that are read as T are identified as being T, fC, caC, or hmC. Performing this type of conversion, such as on a DNA sample as described herein, thus facilitates distinguishing positions containing unmodified C or mC on the one hand from positions containing hmC using the sequence reads obtained from the sample. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA of the first subsample comprises APOBEC-coupled epigenetic (ACE) conversion. In ACE conversion, an AID/APOBEC family DNA deaminase enzyme such as APOBEC3A (A3A) is used to deaminate unmodified cytosine and mC without deaminating hmC, fC, or caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being hmC, fC, or caC positions. Meanwhile, positions that are read as T are identified as being T, unmodified C, or mC. Performing ACE conversion on a DNA sample as described herein thus facilitates distinguishing positions containing hmC from positions containing mC or unmodified C using the sequence reads obtained from the sample. For an exemplary description of ACE conversion, see, e.g., Schutsky et al., Nature Biotechnology 2018; 36: 1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines.

Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the procedure that affects a first nucleobase in the DNA differently from a second nucleobase in the DNA of the first subsample comprises separating DNA originally comprising the first nucleobase from DNA not originally comprising the first nucleobase. In some such embodiments, the first nucleobase is hmC. DNA originally comprising the first nucleobase may be separated from other DNA using a labeling procedure comprising biotinylating positions that originally comprised the first nucleobase. In some embodiments, the first nucleobase is first derivatized with an azide-containing moiety, such as a glucosyl-azide containing moiety. The azide-containing moiety then may serve as a reagent for attaching biotin, e.g., through Huisgen cycloaddition chemistry. Then, the DNA originally comprising the first nucleobase, now biotinylated, can be separated from DNA not originally comprising the first nucleobase using a biotin-binding agent, such as avidin, neutravidin (deglycosylated avidin with an isoelectric point of about 6.3), or streptavidin. An example of a procedure for separating DNA originally comprising the first nucleobase from DNA not originally comprising the first nucleobase is hmC-seal, which labels hmC to form 0-6-azide-glucosyl-5-hydroxymethylcytosine and then attaches a biotin moiety through Huisgen cycloaddition, followed by separation of the biotinylated DNA from other DNA using a biotin-binding agent. For an exemplary description of hmC-seal, see, e.g., Han et al., Mol. Cell 2016; 63: 711-719. This approach is useful for identifying fragments that include one or more hmC nucleobases.

In some embodiments, following such a separation, the method further comprises differentially tagging each of the DNA originally comprising the first nucleobase, the DNA not originally comprising the first nucleobase. The method may further comprise pooling the DNA originally comprising the first nucleobase and the DNA not originally comprising the first nucleobase following differential tagging. The DNA originally comprising the first nucleobase and the DNA not originally comprising the first nucleobase may then be used in downstream analyses. For example, the pooled DNA originally comprising the first nucleobase and the DNA not originally comprising the first nucleobase may be sequenced in the same sequencing cell (such as after being subjected to further treatments, such as those described herein) while retaining the ability to resolve whether a given read came from a molecule of DNA originally comprising the first nucleobase or DNA not originally comprising the first nucleobase using the differential tags.

In some embodiments, the first nucleobase is a modified or unmodified adenine, and the second nucleobase is a modified or unmodified adenine. In some embodiments, the modified adenine is N⁶-methyladenine (mA). In some embodiments, the modified adenine is one or more of N⁶-methyladenine (mA), N⁶-hydroxymethyladenine (hmA), or N⁶-formyladenine (fA).

Techniques comprising partitioning based on methylation status or methylated DNA immunoprecipitation (MeDIP) can be used to separate DNA containing modified bases such as mC, mA, caC (which may be generated by oxidation of mC or hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as mC, caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

In some embodiments, the conversion procedure is an enzymatic conversion procedure which converts the base pairing specificity of modified nucleosides (e.g., DM-seq conversion comprising adding a protective group (such as a carboxymethyl group) to unmodified cytosines, and deaminating 5mC, such as using an APOBEC enzyme) or an enzymatic conversion procedure which converts the base pairing specificity of unmodified nucleosides (such as SEM-seq).

In some cases, the conversion procedure used in the methods of the disclosure is one that changes the base pairing specificity of a modified nucleoside (e.g., methylated cytosine), but does not change the base pairing specificity of the corresponding unmodified nucleoside (e.g., cytosine) or does not change the base pairing specificity of any un-modified nucleoside (e.g., cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as DM-seq may in some cases be preferred over methods such as bisulfite sequencing and EM-seq because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized region of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on % of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g., cytosine), but does not change the base pairing specificity of the corresponding modified nucleoside (e.g., methylated cytosine).

The skilled person can select a suitable method according to their needs, including which nucleoside modifications are to be detected and/or identified.

In some embodiments, the conversion procedure converts modified nucleosides. In some embodiments, the conversion procedure which converts modified nucleosides comprises enzymatic conversion, such as DM-seq, for example, as described in WO023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36: 1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI).

In one embodiment, the methyltransferase enzyme is a variant of M.MpeI having an N374R substitution or an N374K substitution. The methyltransferase can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using βGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (OGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149: 1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH₃CN), lithium borohydride (LiBH₄), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein. In some embodiments, the one or more TET enzymes comprise TETcd. TETcd is described in U.S. Pat. No. 10,260,088 and its sequence is SEQ ID NO: 3 therein. In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker as described, e.g., in U.S. Pat. No. 10,961,525. In some embodiments, the one or more TET enzymes comprise TET1 and TET2. In some embodiments, the one or more TET enzymes comprise a V1900 TET mutant, such as a V1900A, V1900C, V1900G, V19001, or V1900P TET mutant. In some embodiments, the one or more TET enzymes comprise a V1900 TET2 mutant, such as a V1900A, V1900C, V1900G, V19001, or V1900P TET2 mutant. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

### E. Enriching DNA; Enrichment Moieties; Enriched Set

In some embodiments, methods herein comprise enriching (also known as “capturing”) nucleic acid molecules comprising sequences present in a target region set for subsequent analysis. Such enrichment or capture may be performed on any sample or subsample described herein using any suitable approach known in the art. Enriching may be performed on one or more subsamples prepared during methods disclosed herein. In some embodiments, DNA is enriched from at least a first subsample. In some embodiments, DNA is enriched from at least the first subsample or the second subsample, e.g., at least the first subsample and the second subsample. In some embodiments, the enriching is performed after a methylation-preserving amplification step, before or after the DNA is contacted with a methylation-sensitive nuclease and/or a methylation-dependent nuclease, after the DNA is subjected to a procedure that affects a first nucleobase in the DNA differently from a second nucleobase in the DNA, or any combination thereof. Where a first subsample undergoes a separation step (e.g., separating DNA originally comprising the first nucleobase (e.g., hmC) from DNA not originally comprising the first nucleobase, such as hmC-seal), enriching may be performed on any, any two, or all of the DNA originally comprising the first nucleobase (e.g., hmC), the DNA not originally comprising the first nucleobase, and the second subsample. In some embodiments, the subsamples are differentially tagged (e.g., as described herein) and then pooled before undergoing enrichment.

In some embodiments, the enriching comprises contacting the DNA with probes specific for such target regions. In some embodiments, the probes comprise an oligonucleotide and a capture moiety, such as biotin or one or more of the other examples noted below. The probes can have sequences selected to tile across a panel of regions, such as genes.

Methods comprising DNA enrichment using probes comprising a capture moiety, such as target-specific probes labeled with biotin, may also comprise a second moiety or binding partner that binds to the capture moiety, such as streptavidin. In some embodiments, an enrichment moiety and binding partner can have higher and lower capture yields for different sets of probes, such as those used to enrich for (capture) a sequence-variable target region set and an epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. In some embodiments, a capture moiety that is attached to an analyte is captured by its binding partner which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, nonspecifically bound DNA that does not comprise a target region is washed away from the enriched DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, enriched DNA is amplified following elution from the solid support. In some such embodiments, DNA comprising adapters is amplified using PCR primers that anneal to the adapters. In some embodiments, enriched DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, target regions are enriched from an aliquot, portion, or subsample of a sample (e.g., a sample that has undergone attachment of adapters and amplification), while a step of partitioning the DNA may be performed on a separate aliquot, portion, or subsample of the sample.

Enriching for or capturing DNA comprising target regions may comprise contacting the DNA with a first or second set of target-specific probes. Such target-specific probes may have any of the features described herein for sets of target-specific probes, including but not limited to in the embodiments set forth herein and the sections relating to probes herein. Enriching may be performed on a DNA sample or one or more subsamples prepared during methods disclosed herein. In some embodiments, DNA is enriched from a first subsample or a second subsample, such as after methylation-preserving amplification or after subjecting the DNA, or a subsample thereof, to a procedure that affects a first nucleobase in the DNA differently from a second nucleobase in the DNA. In some embodiments, the subsamples are differentially tagged (e.g., as described herein) and then pooled before undergoing enrichment. Exemplary methods for enriching DNA comprising epigenetic and/or sequence-variable target regions can be found in, e.g., WO 2020/160414, which is hereby incorporated by reference.

The enriching step or steps may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization.

In some embodiments, methods described herein comprise enriching for a plurality of target region sets of cfDNA obtained from a subject. The target regions may comprise differences depending on whether they originated from a tumor or from healthy cells or from a certain cell type. For example, the target regions comprising epigenetic target regions may show differences in methylation levels and/or fragmentation patterns depending on whether they originated from a tumor or from healthy cells. Similarly, the target regions comprising sequence-variable target regions may show differences in sequence depending on whether they originated from a tumor or from healthy cells. The enriching step produces an enriched set of cfDNA molecules. In some embodiments, cfDNA molecules corresponding to a sequence-variable target region set are enriched at a greater capture yield in the enriched set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to enrich DNA corresponding to the sequence-variable target region set at a greater capture yield than DNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation.

In some embodiments, the enriched DNA is amplified. In various embodiments, the methods further comprise sequencing the enriched DNA, e.g., to different degrees of sequencing depth for the epigenetic and sequence-variable target region sets, consistent with the discussion herein. In some embodiments, RNA probes are used. In some embodiments, DNA probes are used. In some embodiments, single stranded probes are used. In some embodiments, double stranded probes are used. In some embodiments, single stranded RNA probes are used. In some embodiments, double stranded DNA probes are used.

In some embodiments, an enriching step is performed with probes for a sequence-variable target region set and probes for an epigenetic target region set in the same vessel at the same time, e.g., the probes for the sequence-variable and epigenetic target region sets and capture probes are in the same composition. This approach provides a relatively streamlined workflow.

Alternatively, the enriching step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising enriched DNA corresponding to the sequence-variable target region set and enriched DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the enriching step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes (e.g., a “sample index”), are included in the enriched DNA, such as during a post-enrichment amplification step. In some embodiments, such tags are referred to as “supplemental adapters.” Tags can facilitate identification of the origin of a nucleic acid. For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

In some embodiments, a collection of target-specific probes is used in methods comprising enriched DNA described herein. In some embodiments, the collection of target-specific probes comprises target-binding probes specific for one or more target region sets. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

### 1. Enriched Set

In some embodiments, an enriched (also known as captured) set of DNA (e.g., cfDNA) is provided. With respect to the disclosed methods, the enriched set of DNA may be provided, e.g., by performing an enriching step, such as after methylation-preserving amplification or after subjecting the DNA, or a subsample thereof, to a procedure that affects a first nucleobase in the DNA differently from a second nucleobase in the DNA. The enriched set may comprise DNA corresponding to a sequence-variable target region set, an epigenetic target region set, or a combination thereof.

In some embodiments, a first target region set is enriched from the DNA or a subsample thereof (such as a first subsample), comprising at least epigenetic target regions. The epigenetic target regions enriched from the DNA or a subsample thereof may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that show lower methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypermethylation variable target regions in the DNA or a subsample thereof can be an indicator of the presence (or recurrence, depending on the history of the subject) of cancer.

In some embodiments, a second target region set is enriched from the DNA or a subsample thereof (such as a second subsample), comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the DNA or a subsample thereof (such as a second subsample) can be an indicator of the presence (or recurrence, depending on the history of the subject) of cancer.

In some embodiments the quantity of enriched sequence-variable target region DNA is greater than the quantity of the enriched epigenetic target region DNA, when normalized for the difference in the size of the targeted regions (footprint size).

Alternatively, first and second enriched sets may be provided, comprising, respectively, DNA corresponding to a sequence-variable target region set and DNA corresponding to an epigenetic target region set. The first and second enriched sets may be combined to provide a combined enriched set.

In some embodiments in which an enriched set comprising DNA corresponding to the sequence-variable target region set and the epigenetic target region set includes a combined enriched set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

F. Contacting DNA with a Methylation-Sensitive or Methylation-Dependent Nuclease

In some embodiments, DNA or a subsample thereof (e.g., a first, second, or third subsample prepared by partitioning a sample as described herein, such as on the basis of a level of a cytosine modification, such as methylation, e.g., 5-methylation) is contacted with a methylation-dependent nuclease or methylation-sensitive nuclease. The contacting can be performed, for example, using a sample that has been partitioned into a plurality of subsamples as disclosed herein. Unless otherwise indicated, where partitioning is performed on the basis of a cytosine modification, the first subsample is the subsample with a higher level of the modification; the second subsample is the subsample with a lower level of the modification; and, when present, the third subsample has a level of the modification intermediate between the first and second subsamples.

In some embodiments, methods herein comprise contacting DNA with a methylation-sensitive nuclease, thereby degrading DNA comprising unmethylated sequences or sequences having low levels of methylation. In some such embodiments, the methylation-sensitive nuclease is a methylation-sensitive restriction enzyme (MSRE), thereby degrading DNA comprising an unmethylated recognition site of the MSRE. Methylation-sensitive nucleases can thus be used in methods herein comprising one or more steps that deplete unmodified or unmethylated sequences, such as those that are prevalent in cfDNA from a subject.

In some embodiments, methods herein comprise contacting DNA with a methylation-dependent nuclease, thereby degrading DNA comprising methylated sequences or sequences having high levels of methylation. In some such embodiments, the methylation-dependent nuclease is a methylation-dependent restriction enzyme (MDRE), thereby degrading DNA comprising an methylated recognition site of the MSRE. Methylation-dependent nucleases can thus be used in methods herein comprising one or more steps that deplete modified or methylated sequences, such as those that are prevalent in cfDNA from a subject.

As discussed above, partitioning procedures may result in imperfect sorting of DNA molecules among the subsamples. The choice of a methylation-dependent nuclease or methylation-sensitive nuclease can be made so as to degrade nonspecifically partitioned DNA. For example, the second subsample can be contacted with a methylation-dependent nuclease, such as a methylation-dependent restriction enzyme. This can degrade nonspecifically partitioned DNA in the second subsample (e.g., methylated DNA) to produce a treated second subsample. Alternatively or in addition, the first subsample can be contacted with a methylation-sensitive endonuclease, such as a methylation-sensitive restriction enzyme, thereby degrading nonspecifically partitioned DNA in the first subsample to produce a treated first subsample. Degradation of nonspecifically partitioned DNA in either or both of the first or second subsamples is proposed as an improvement to the performance of methods that rely on accurate partitioning of DNA on the basis of a cytosine modification, e.g., to detect the presence of aberrantly modified DNA in a sample, to determine the tissue of origin of DNA, and/or to determine whether a subject has cancer. For example, such degradation may provide improved sensitivity and/or simplify downstream analyses. In general, where nonspecifically partitioned DNA would be hypermethylated, such as in a hypomethylated partition, a methylation-dependent nuclease, such as a methylation-dependent restriction enzyme, should be used. Conversely, where nonspecifically partitioned DNA would be hypomethylated, such as in a hypermethylated partition, a methylation-sensitive nuclease, such as a methylation-sensitive restriction enzyme, should be used. Methylation-dependent nucleases, such as methylation-dependent restriction enzymes, preferentially cut methylated DNA relative to unmethylated DNA, while methylation-sensitive nucleases, such as methylation-sensitive restriction enzymes, preferentially cut unmethylated DNA relative to methylated DNA.

In a contacting a subsample with a nuclease, one or more nucleases can be used. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting the second subsample with more than one methylation-dependent restriction enzyme can more completely degrade nonspecifically partitioned hypermethylated DNA. Similarly, contacting the first subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used. In some embodiments, the methylation-dependent nuclease comprises FspEI. In some embodiments, the methylation-dependent nuclease comprises FspEI and MspJI, e.g., used sequentially.

In some embodiments, a methylation-sensitive nuclease comprises one or more of AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, FspEI is used for digesting the nucleic acid molecules in at least one subsample (e.g., a hypomethylated partition). In some embodiments, BstUI, HpaII and Hin6I are used for digesting the nucleic acid molecules in at least one subsample (e.g., a hypermethylated partition) and FspEI is used for digesting the nucleic acid molecules in at least one other subsample (e.g., a hypomethylated partition). In embodiments involving an intermediately methylated partition, the nucleic acid molecules therein may be digested with a methylation-sensitive nuclease or a methylation-dependent nuclease. In some embodiments, the nucleic acid molecules in an intermediately methylated partition are digested with the same nuclease(s) as the hypermethylated partition. For example, the intermediately methylated partition may be pooled with the hypermethylated partition and then the pooled partitions may be subjected to digestion. In some embodiments, the nucleic acid molecules in an intermediately methylated partition are digested with the same nuclease(s) as the hypomethylated partition. For example, the intermediately methylated partition may be pooled with the hypomethylated partition and then the pooled partitions may be subjected to digestion.

In some embodiments, a subsample is contacted with a nuclease as described above after a step of tagging or attaching adapters to both ends of the DNA. The tags or adapters can be resistant to cleavage by the nuclease using any of the approaches described above. In this approach, cleavage can prevent the nonspecifically partitioned molecule from being carried through the analysis because the cleavage products lack tags or adapters at both ends.

Alternatively, a step of tagging or attaching adapters can be performed after cleavage with a nuclease as described above. Cleaved molecules can be then identified in sequence reads based on having an end (point of attachment to tag or adapter) corresponding to a nuclease recognition site. Processing the molecules in this way can also allow the acquisition of information from the cleaved molecule, e.g., observation of somatic mutations. When tagging or attaching adapters after contacting the subsample with a nuclease, and low molecular weight DNA such as cfDNA is being analyzed, it may be desirable to remove high molecular weight DNA (such as contaminating genomic DNA) from the sample before the contacting step. It may also be desirable to use nucleases that can be heat-inactivated at a relatively low temperature (e.g., 65° C. or less, or 60° C. or less) to avoid denaturing DNA, in that denaturation may interfere with subsequent ligation steps.

Where a sample is partitioned into three subsamples, including a third subsample containing intermediately methylated molecules, the third subsample is in some embodiments contacted with a methylation-sensitive nuclease. Such a step may have any of the features described elsewhere herein with respect to contacting steps, and may be performed before or after a step of tagging or attaching adapters as discussed above. In some embodiments, the first and third subsamples are combined before being contacted with a methylation-sensitive nuclease. Such a step may have any of the features described elsewhere herein with respect to contacting steps, and may be performed before or after a step of tagging or attaching adapters as discussed above. In some embodiments, the first and third subsamples are differentially tagged before being combined.

Alternatively, where a sample is partitioned into three subsamples, including a third subsample containing intermediately methylated molecules, the third subsample is in some embodiments contacted with a methylation-dependent nuclease. Such a step may have any of the features described elsewhere herein with respect to contacting steps, and may be performed before or after a step of tagging or attaching adapters as discussed above. In some embodiments, the second and third subsamples are combined before being contacted with a methylation-dependent nuclease. Such a step may have any of the features described elsewhere herein with respect to contacting steps, and may be performed before or after a step of tagging or attaching adapters as discussed above. In some embodiments, the second and third subsamples are differentially tagged before being combined.

In some embodiments, the DNA is purified after being contacted with the nuclease, e.g., using SPRI beads. Such purification may occur after heat inactivation of the nuclease. Alternatively, purification can be omitted; thus, for example, a subsequent step such as amplification can be performed on the subsample containing heat-inactivated nuclease. In another embodiment, the contacting step can occur in the presence of a purification reagent such as SPRI beads, e.g., to minimize losses associated with tube transfers. After cleavage and heat inactivation, the SPRI beads can be re-used for cleanup by adding molecular crowding reagents (e.g., PEG) and salt.

G. Partitioning the Sample into a Plurality of Subsamples

Disclosed methods herein comprise analyzing DNA in a sample. In such methods, different forms of DNA (e.g., hypermethylated and hypomethylated DNA) can be physically partitioned based on one or more characteristics of the DNA into a plurality of subsamples. This approach can be used to determine, for example, whether certain sequences are hypermethylated or hypomethylated. Such partitioning can be performed before or after a step of methylation-preserving amplification, and before sequencing. Partitioning can be performed using a DNA sample or one or more subsamples thereof, as described herein.

In some embodiments, the partitioning comprises contacting the DNA with an agent that recognizes a modification associated with (e.g., in) the DNA. In some embodiments, the agent that recognizes the modification is an antibody. In some embodiments, the agent is immobilized on a solid support. In some embodiments, the partitioning comprises immunoprecipitation, e.g., using the antibody agent, such as an antibody, immobilized on solid support.

In some embodiments, the modification is methylation, and in some such embodiments, the partitioning comprises partitioning on the basis of methylation level. In some such embodiments, the agent is a methyl binding reagent. In some embodiments, the methyl binding reagent specifically recognizes 5-methylcytosine. In some such embodiments, the agent is a hydroxymethyl binding reagent. In some embodiments, the methyl binding reagent specifically recognizes 5-hydroxymethylcytosine, biotinylated 5-hydroxymethylcytosine, glucosylated 5-hydroxymethylcytosine, or sulfonylated 5-hydroxymethylcytosine. In some embodiments, the partitioning comprises partitioning on the basis of binding to a protein comprising contacting the sample comprising the DNA with a binding reagent specific for the protein. In some such embodiments, binding reagent specifically binds a methylated protein, an acetylated protein, such as a methylated or acetylated histone. In some embodiments, the binding reagent specifically binds an unmethylated or unacetylated protein epitope.

In some embodiments, the modification is hydroxymethylation, and in some such embodiments, the partitioning comprises partitioning on the basis of hydroxymethylation level. In some such embodiments, the agent is a hydroxymethyl binding reagent, such as an antibody. In some embodiments, the hydroxymethyl binding reagent (e.g., antibody) specifically recognizes 5-hydroxymethylcytosine (5-hmC). In some embodiments, a modification such as hydroxymethylation is labeled (e.g., biotinylated, glucosylated, or sulfonated) before being contacted with an agent that recognizes the labeled form of the modification. For example, 5-hmC can be enzymatically glucosylated and then partitioned based on binding to J-binding protein 1. Exemplary methods of labeling and/or partitioning 5-hmC are provided, e.g., in Song et al., Nat. Biotech. 29:68-72 (2010); Ko et al., Nature 468:839-843 (2010); and Robertson et al., Nucleic Acids Res. 39:e55 (2011).

Where immunoprecipitation is used and involves an antibody that recognizes single-stranded DNA, the DNA may be converted to double-stranded form by complementary strand synthesis, e.g., before a degrading step. Such synthesis may use an adapter as a primer binding site, or can use random priming.

In some embodiments, a sample comprising DNA is partitioned into a plurality of subsamples. In some embodiments, the plurality of partitioned subsamples comprises two subsamples, a first subsample and a second subsample. In some embodiments, the plurality of partitioned subsamples comprises three subsamples, a first subsample, second subsample, and third subsample. In some embodiments, the methods comprise a partitioning step that is performed prior to the sequencing and (a) prior to performing the methylation-preserving amplification of the DNA, (b) after performing the methylation-preserving amplification of the DNA, (c) prior to the enriching for one or more sets of epigenetic target regions of DNA from the DNA; and/or (d) after the enriching for one or more sets of epigenetic target regions of DNA from the DNA.

In some embodiments comprising a third partitioned subsample, the third subsample comprises DNA associated with a modification in a greater proportion than it is associated with DNA in the second subsample and in a lesser proportion that it is associated with DNA in the first subsample.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable target regions are analyzed to determine whether they show differential methylation characteristic of tumor cells or cells of a type that does not normally contribute to the DNA sample being analyzed (such as cfDNA), and/or particular immune cell types.

In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as copy number variations (CNVs), single nucleotide variations (SNVs), insertions/deletions (indels), and/or fusions in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Exemplary partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as HpaII, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents or binding reagents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner® Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning is performed after contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed herein.

Examples of agents that recognize a modified nucleobase contemplated herein include, but are not limited to:
(a) MeCP2 and MBD2 are proteins that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DIU1.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads. In some embodiments, the partitions of DNA are desalted and concentrated in preparation for enzymatic steps of library preparation.

H. Pooling DNA from at Least a First and Second Subsamples or Portions Thereof.

In some embodiments, such as following a partitioning step, the methods comprise preparing a pool comprising at least a portion of the DNA of the second subsample (also referred to as the hypomethylated partition) and at least a portion of the DNA of the first subsample (also referred to as the hypermethylated partition). Target regions, e.g., including epigenetic target regions and/or sequence-variable target regions, may be enriched from the pool. The steps of enriching for a target region set from at least a portion of a subsample described elsewhere herein encompass enrichment steps performed on a pool comprising DNA from the first and second subsamples. A step of amplifying DNA in the pool may be performed before enriching for target regions from the pool. The enriching step may have any of the features described elsewhere herein.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target-regions from the hypermethylated and hypomethylated partitions and epigenetic target regions from the hypermethylated partition. As such, in methods where sequence-variable target-regions and epigenetic target regions are being enriched, the latter may be enriched to a lesser extent than one or more of the sequence-variable target-regions from the hypermethylated and hypomethylated partitions and epigenetic target regions from the hypermethylated partition. For example, sequence-variable target regions can be enriched from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of the hypomethylated partition, e.g., less than about 50% of the DNA of the hypomethylated partition, such as less than or equal to about 45%, 40%, 35%, 30%, 25%, 20%, 15%, 10%, or 5% of the DNA of the hypomethylated partition. In some embodiments, the pool comprises about 5%-25% of the DNA of the hypomethylated partition. In some embodiments, the pool comprises about 10%-20% of the DNA of the hypomethylated partition. In some embodiments, the pool comprises about 10% of the DNA of the hypomethylated partition. In some embodiments, the pool comprises about 15% of the DNA of the hypomethylated partition. In some embodiments, the pool comprises about 20% of the DNA of the hypomethylated partition.

In some embodiments, the pool comprises a portion of the hypermethylated partition, which may be at least about 50% of the DNA of the hypermethylated partition. For example, the pool may comprise at least about 55%, 60%, 65%, 70%, 75%, 80%, 85%, 90%, or 95% of the DNA of the hypermethylated partition. In some embodiments, the pool comprises 50-55%, 55-60%, 60-65%, 65-70%, 70-75%, 75-80%, 80-85%, 85-90%, 90-95%, or 95-100% of the DNA of the hypermethylated partition. In some embodiments, the second pool comprises all or substantially all of the hypermethylated partition.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise enriching for at least a first set of target regions from the first pool, e.g., wherein the first pool is as set forth in any of the embodiments above. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this enrichment step. In some embodiments, enriching for the first set of target regions from the first pool comprises contacting the DNA of the first pool with a first set of target-specific probes. In some embodiments, the first set of target-specific probes comprises target-binding probes specific for the sequence-variable target regions. In some embodiments, the first set of target-specific probes comprises target-binding probes specific for the sequence-variable target regions, hypomethylation variable target regions and/or fragmentation variable target regions.

In some embodiments, the methods comprise enriching for a second set of target regions or plurality of sets of target regions from the second pool, e.g., wherein the first pool is as set forth in any of the embodiments above. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this enrichment step. In some embodiments, enriching for the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is enriched from the second pool but not from the first pool. In some embodiments, a plurality of hypermethylation variable target regions are enriched from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

### I. Detecting; Sequencing

In some embodiments, detection of the presence or absence of DNA sequences and/or modifications comprises sequencing. In some embodiments, the DNA is sequenced in a manner that distinguishes a first nucleobase from a second nucleobase. In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, long-read sequencing (also known as single-molecule sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), next generation sequencing (NGS), Single Molecule Sequencing by Synthesis (SMSS) (Helicos), enzymatic methyl sequencing (EM-Seq), Tet-assisted pyridine borane sequencing (TAPS), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms. Sequencing reactions can be performed in a variety of sample processing units, which may include multiple lanes, multiple channels, multiple wells, or other mean of processing multiple sample sets substantially simultaneously. Sample processing units can also include multiple sample chambers to enable processing of multiple runs simultaneously.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine (Weirather J L, et al., “Comprehensive comparison of Pacific Biosciences and oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: 10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil- and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, the sequencing comprises targeted sequencing in which one or more genomic regions of interest are sequenced. In some such embodiments, the genomic regions of interest comprise regions present one or more genes selected from Tables 1, 2, 3, 4, and/or 5. In some such embodiments, DNA sequences that do not comprise regions of interest are not sequenced. Some embodiments comprise non-targeted sequencing, e.g., all genomic regions of the DNA in a treated sample or subsample are sequenced, or genomic regions are randomly chosen for sequencing. In other embodiments, detecting DNA the presence or absence of sequences comprises sequencing DNA that is not enriched for genomic regions of interest (non-targeted sequencing), e.g., wherein detectable sequences are obtained in a substantially unbiased manner.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or at most 5000, 2500, 1000, 500 or 100 different genes.

Simultaneous sequencing reactions may be performed using multiplex sequencing. In some embodiments, cell-free nucleic acids may be sequenced with at least 1000, 2000, 3000, 4000, 5000, 6000, 7000, 8000, 9000, 10000, 50000, 100,000 sequencing reactions. In other embodiments, cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

In some embodiments, sequences that were not cleaved during the degrading step are sequenced. In some embodiments, less than 50%, 40%, 30%, 20%, 10%, 5%, 4%, 3%, 2%, or 1% of sequences that were cleaved during the degrading step are sequenced.

### J. Subjects

In some embodiments, the DNA (e.g., cfDNA or DNA from a sample comprising cells) is obtained from a subject (e.g., a test subject) having a cancer or a precancer. In some embodiments, the subject has a stage I cancer, stage II cancer, stage III cancer, or stage IV cancer. In some embodiments, the DNA from the subject is obtained and/or derived from a sample obtained from the subject. In some embodiments, the DNA is obtained from a subject suspected of having a cancer or a precancer. In some embodiments, the DNA is obtained from a subject having a tumor. In some embodiments, the DNA is obtained from a subject suspected of having a tumor. In some embodiments, the DNA is obtained from a subject having neoplasia. In some embodiments, the DNA is obtained from a subject suspected of having neoplasia. In some embodiments, the DNA is obtained from a subject in remission from a tumor, cancer, or neoplasia (e.g., following chemotherapy, surgical resection, radiation, or a combination thereof). In any of the foregoing embodiments, the precancer, cancer, tumor, or neoplasia or suspected precancer, cancer, tumor, or neoplasia may be of the bladder, head or neck, lung, colon, rectum, kidney, breast, prostate, skin, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject. In any of the foregoing embodiments, the subject may be a test subject.

### K. Samples

A sample can be any biological sample isolated from a subject. A sample can be a bodily sample. Samples can include body tissues or fluids, such as known or suspected solid tumors (such as carcinomas, adenocarcinomas, or sarcomas), whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, gingival crevicular fluid, bone marrow, pleural effusions, pleura fluid, cerebrospinal fluid, saliva, mucous, sputum, semen, sweat, and urine. Samples are preferably body fluids, particularly blood and fractions thereof, cerebrospinal fluid, pleura fluid, saliva, sputum, or urine. A sample can be in the form originally isolated from a subject or can have been subjected to further processing to remove or add components, such as cells, or enrich for one component relative to another.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, post-translation modifications (PTMs) of chromatin, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 mL, 5-20 mL, 10-20 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be 5 to 20 mL. In some embodiments, the sample volume is 3-5 mL of plasma, such as 4 mL of plasma, per 10 mL whole blood.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

In some embodiments, the sample comprises buffy coat separated from whole blood. Exemplary volumes of sampled buffy coat are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled buffy coat may be 1 to 10 mL. In some embodiments, the sample volume is 0.1-0.5 mL of buffy coat, such as 0.3 mL of buffy coat, per 10 mL whole blood.

In some embodiments, the sample comprises PBMCs separated from whole blood. Exemplary volumes of sampled PBMCs are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled PBMCs may be 1 to 10 mL. In some embodiments, the sample volume is 0.1-0.5 mL of PBMCs, such as 0.3 mL of PBMCs, per 10 mL whole blood.

In some embodiments, the sample comprises leukocytes separated from subject blood using leukapheresis. Exemplary volumes of sampled leukocytes from leukapheresis are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 10 mL, or 20 mL. A volume of sampled leukocytes from leukapheresis may be 1 to 10 mL. In some embodiments, the sample volume is 0.1-0.6 mL of leukocytes from leukapheresis, such as 0.4 mL of leukocytes, per 10 mL whole blood.

A sample can comprise various amount of nucleic acid that contains genome equivalents. For example, a sample of about 30 ng DNA can contain about 10,000 (10⁴) haploid human genome equivalents. Similarly, a sample of about 100 ng of DNA can contain about 30,000 haploid human genome equivalents.

A sample can comprise nucleic acids from different sources, e.g., from cells of the same subject or from cells of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., precancer cells or cancer cells. A sample can comprise DNA carrying cancer-associated mutations (e.g., cancer-associated somatic mutations). A sample can comprise an epigenetic variant (i.e., a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of nucleic acids (e.g., DNA from a buffy coat sample or any other sample comprising cells, such as a blood sample (e.g., a whole blood sample, a leukapheresis sample, or a PBMC sample) in a sample before amplification range from about 1 fg to about 1 g, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of nucleic acid molecules. The method can comprise obtaining 1 femtogram (fg) to 200 ng.

Nucleic acids can be isolated from cells, such as cells of bodily fluids. Cells can be lysed and cellular nucleic acids processed. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA, and single stranded RNA. In some embodiments, single stranded DNA and RNA can be converted to double stranded forms so they are included in subsequent processing and analysis steps.

Reference or control molecules can be added to or spiked into a sample as a control or normalization standard. For example, a certain amount of modified DNA from a species other than the species of the subject from which the sample was obtained or synthetic nucleic acids comprising certain modifications may be added to the sample. In some embodiments, the reference or control molecules are distinguishable from the molecules originally present in the sample. In some embodiments, the detected DNA sequences are normalized to the reference or control molecules.

DNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### L. Analysis

The present disclosure provides methods of analyzing DNA using a methylation-preserving amplification step. In some embodiments, the disclosed methods comprise analyzing DNA (such as DNA from a subject) to identify at least one cell type, cell cluster type, tissue type, and/or cancer type from which one or more type-specific epigenetic target regions and/or type-specific sequence-variable target regions originated. In some embodiments, methods comprise determining the level of one or more type-specific epigenetic target regions and/or type-specific sequence-variable target regions that originated from the at least one cell type, cell cluster type, tissue type, and/or cancer type.

An exemplary method for analyzing DNA comprises the following steps (e.g., in the order listed below), which is illustrated in in FIG. 1A:
1. Preparing an extracted DNA sample (e.g., extracting DNA, such as cfDNA, from a human sample, such as a blood sample).
2. Subjecting the extracted DNA to library preparation, including attaching tags (e.g., adapters) comprising molecular barcodes, which are optionally methylated.
3. Performing a methylation-preserving amplification (also referred to as a methylation maintaining amplification).
4. Performing either steps 4A-1 and 4A-2, or steps 4B-1 and 4B-2:
4A-1. Subjecting the DNA from step 3 to a procedure that affects a first nucleobase in the DNA differently from a second nucleobase in the DNA, such as any form of epigenetic base conversion described elsewhere herein, e.g., bisulfite conversion.
4A-2. Enriching the DNA from step 4A-1 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-1. Enriching the DNA from step 3 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-2. Subjecting the enriched DNA to a procedure that affects a first nucleobase in the DNA differently from a second nucleobase in the DNA, such as any form of epigenetic base conversion described elsewhere herein, e.g., bisulfite conversion.
5. Sequencing the DNA via NGS.
6. Performing bioinformatics analysis of the NGS data, comprising using one or more components (e.g., a barcode or set of barcodes in combination with a genomic start site and/or a genomic stop site) of each read to identify and group reads into molecular families (e.g., DNA molecules), wherein the reads within a molecular family have (a) a common molecular barcode and (b) the same genomic start position, (c) the same genomic stop position, or (d) both (b) and (c).
7. Determining a consensus epigenetic sequence (methylation status) for each cytosine in the sequence of a given molecular family.

Another exemplary method for analyzing DNA comprises the following steps (e.g., in the order listed below), of which steps 3, 5, and 8-11 are illustrated in FIG. 1A:
1. Preparing an extracted DNA sample (e.g., extracting DNA, such as cfDNA, from a human sample, such as a blood sample).
2. Subjecting the extracted DNA to library preparation, including attaching adapters comprising molecular barcodes, which are optionally methylated. An adapter further comprises a restriction enzyme cleavage site between the barcode and at least a portion of the remainder of the adapter. In such cases, the barcode is located between the DNA sequence to be analyzed and the restriction site. For example, in an adapter ligated to the 5′ end of a DNA sequence, the barcode is 3′ of the restriction site and 5′ of the DNA sequence to be analyzed. Further, a barcode in this example does not comprise cytosines in a non-CpG context.
3. Performing a methylation-preserving amplification (also referred to as a methylation maintaining amplification).
4. Contacting the amplified DNA with a restriction enzyme that recognizes the restriction site in the adapter, to remove at least a portion of the adapter that does not comprise the barcode. In some embodiments, cleavage of the DNA by the restriction enzyme results in an overhang (such as an A or T overhang), e.g., that can be used as a sticky end for a later ligation.
5. Subjecting the DNA from step 3 to a procedure that affects a first nucleobase in the DNA differently from a second nucleobase in the DNA, wherein the first nucleobase is an unmethylated cytosine and the subjecting step affects the unmethylated cytosine (e.g., bisulfite conversion, EM-Seq, or SEM-Seq).
6. Attaching supplemental adapters to the DNA, wherein the supplemental adapters do not comprise barcodes.
7. Performing a uracil- and/or dihydrouracil-tolerant amplification.
8. Enriching the DNA from step 8 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA, wherein the amplification comprises differentially tagging DNA enriched for, e.g., one or more epigenetic target region sets or for one or more sequence variable target region sets. The differential tagging can comprise attaching sample indices to the DNA.
9. Sequencing the DNA via NGS.
10. Performing bioinformatics analysis of the NGS data, comprising using one or more components (e.g., a barcode or set of barcodes in combination with a genomic start site and/or a genomic stop site) of each read to identify and group reads into molecular families (e.g., DNA molecules), wherein the reads within a molecular family have (a) a common molecular barcode and (b) the same genomic start position, (c) the same genomic stop position, or (d) both (b) and (c).
11. Determining a consensus epigenetic sequence (methylation status) for each cytosine in the sequence of a given molecular family.

Another exemplary method for analyzing DNA comprises the following steps (e.g., in the order listed below), which is illustrated in FIG. 1B:
1. Preparing an extracted DNA sample (e.g., extracting DNA, such as cfDNA, from a human sample, such as a blood sample).
2. Subjecting the extracted DNA to library preparation, optionally including attaching tags (e.g., adapters) comprising molecular barcodes, which are optionally methylated.
3. Performing a linear methylation-preserving amplification (also referred to as a methylation maintaining amplification), e.g., by providing a reverse primer as the only primer.
4. Performing either steps 4A-1 and 4A-2, or steps 4B-1 and 4B-2:
4A-1. Subjecting the extracted DNA to a procedure that affects a first nucleobase in the DNA differently from a second nucleobase in the DNA, such as any form of epigenetic base conversion described elsewhere herein, e.g., bisulfite conversion.
4A-2. Enriching the DNA from step 4A-1 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-1. Enriching the DNA from step 3 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-2. Subjecting the enriched DNA to a procedure that affects a first nucleobase in the DNA differently from a second nucleobase in the DNA, such as any form of epigenetic base conversion described elsewhere herein, e.g., bisulfite conversion.
5. Sequencing the DNA via NGS.
6. Performing bioinformatics analysis of the NGS data, comprising using one or more components (e.g., a barcode or set of barcodes in combination with a genomic start site and/or a genomic stop site) of each read to identify and group reads into molecular families (e.g., DNA molecules), wherein the reads within a molecular family have (a) a common molecular barcode and (b) the same genomic start position, (c) the same genomic stop position, or (d) both (b) and (c).
7. Determining a consensus epigenetic sequence (methylation status) for each cytosine in the sequence of a given molecular family.

Another exemplary method for analyzing DNA comprises the following steps (e.g., in the order listed below), which is illustrated in FIG. 1B:
1. Preparing an extracted DNA sample (e.g., extracting DNA, such as cfDNA, from a human sample, such as a blood sample).
2. Subjecting the extracted DNA to library preparation, including attaching adapters comprising molecular barcodes.
3. Performing a methylation-preserving linear amplification.
4. Performing either steps 4A-1 and 4A-2, or steps 4B-1 and 4B-2:
4A-1. Subjecting the DNA from step 3 to a procedure that affects a first nucleobase in the DNA differently from a second nucleobase in the DNA, such as any form of epigenetic base conversion described elsewhere herein, e.g., bisulfite conversion.
4A-2. Enriching the DNA from step 4A-1 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-1. Enriching the DNA from step 3 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA.
4B-2. Subjecting the enriched DNA to a procedure that affects a first nucleobase in the DNA differently from a second nucleobase in the DNA, such as any form of epigenetic base conversion described elsewhere herein, e.g., bisulfite conversion.
5. Sequencing the DNA via NGS.
6. Performing bioinformatics analysis of the NGS data, comprising using one or more components (e.g., a barcode or set of barcodes, a genomic start site, and/or a genomic stop site) of each read to identify and group NGS reads into molecular families (e.g., DNA molecules), wherein the reads within a molecular family have (a) a common molecular barcode, (b) the same genomic start position, (c) the same genomic stop position, or a combination of (a) with (b) and/or (c).
7. Determining a consensus epigenetic sequence (methylation status) for each cytosine/base in the sequence of a given molecular family.

Another exemplary method for analyzing DNA comprises the following steps (e.g., in the order listed below), of which steps 3, 5, and 8-11 are illustrated in FIG. 1B:
1. Preparing an extracted DNA sample (e.g., extracting DNA, such as cfDNA, from a human sample, such as a blood sample).
2. Subjecting the extracted DNA to library preparation, including attaching adapters comprising molecular barcodes. An adapter further comprises a restriction enzyme cleavage site between the barcode and at least a portion of the remainder of the adapter. In such cases, the barcode is located between the DNA sequence to be analyzed and the restriction site. For example, in an adapter ligated to the 5′ end of a DNA sequence, the barcode is 3′ of the restriction site and 5′ of the DNA sequence to be analyzed. Further, a barcode in this example does not comprise cytosines in a non-CpG context.
3. Performing a methylation-preserving linear amplification.
4. Contacting the amplified DNA with a restriction enzyme that recognizes the restriction site in the adapter, to remove at least a portion of the adapter that does not comprise the barcode. In some embodiments, cleavage of the DNA by the restriction enzyme results in an overhang (such as an A or T overhang), e.g., that can be used as a sticky end for a later ligation.
5. Subjecting the DNA from step 3 to a procedure that affects a first nucleobase in the DNA differently from a second nucleobase in the DNA, wherein the first nucleobase is an unmethylated cytosine and the subjecting step affects the unmethylated cytosine (e.g., bisulfite conversion, EM-Seq, or SEM-Seq).
6. Attaching supplemental adapters to the DNA, wherein the supplemental adapters do not comprise barcodes.
7. Performing a uracil- and/or dihydrouracil-tolerant amplification.
8. Enriching the DNA from step 8 for nucleic acid molecules comprising sequences present in one or more target region sets (such as an epigenetic target region set and/or a sequence variable target region set, which may respectively comprise any one or more of the epigenetic target region sets and sequence variable target region sets described elsewhere herein), and optionally amplifying the enriched DNA, wherein the amplification comprises differentially tagging DNA enriched for, e.g., one or more epigenetic target region sets or for one or more sequence variable target region sets. The differential tagging can comprise attaching sample indices to the DNA.
9. Sequencing the DNA via NGS.
10. Performing bioinformatics analysis of the NGS data, comprising using one or more components (e.g., a barcode or set of barcodes, a genomic start site, and/or a genomic stop site) of each read to identify and group NGS reads into molecular families (e.g., DNA molecules), wherein the reads within a molecular family have (a) a common molecular barcode, (b) the same genomic start position, (c) the same genomic stop position, or a combination of (a) with (b) and/or (c).
11. Determining a consensus epigenetic sequence (methylation status) for each cytosine/base in the sequence of a given molecular family.

In some embodiments, the consensus epigenetic sequence (methylation status) for each cytosine/base in sequence of a given molecular family can be determined based on a quantitative measure obtained from the reads (within the family) indicative of being methylated or not. In some embodiments, if the quantitative measure exceeds a family methylation level threshold, then the base can be considered as being methylated. Family methylation level threshold for calling a given base methylated should be informed by methyltransferase fidelity (i.e., sensitivity and/or specificity) and number of amplification cycles, if amplification is PCR or more generally exponential/quasi-exponential in nature (DNA copies can be templates for subsequent DNA amplification).

In some embodiments, detecting the presence, levels, or absence of DNA sequences and/or modifications facilitates disease diagnosis or identification of appropriate treatments. In some embodiments, the presence of or a change in the levels of one or more sequences and/or modifications is indicative of the presence of a disease or disorder in a subject, such as cancer or precancer, or other disorder that causes changes in nucleic acids relative to a healthy subject.

Further, the present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. In some embodiments, the condition is cancer or precancer. In some embodiments, the condition is characterized (e.g., staging cancer or determining heterogeneity of a cancer), response to treatment of a condition is monitored, or prognosis risk of developing a condition or subsequent course of a condition is determined. The present disclosure can also be useful in determining the efficacy of a particular treatment option. Successful treatment options may decrease the amount of detected DNA sequences associated with a cancer in a subject's blood as there may be fewer cancer cells to shed DNA. In other examples, this may not occur. In another example, certain treatment options may be correlated with genetic profiles of cancers over time. This correlation may be useful in selecting a therapy

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, colon cancers, prostate cancers, thyroid cancers, bladder cancers, head and neck cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In some embodiments, a method described herein comprises detecting the presence or absence of nucleic acids, such as DNA, produced by a tumor (or neoplastic cells, or cancer cells) or by precancer cells.

Information and data generated by the methods disclosed herein can also be used for characterizing a specific form of cancer. Cancers are often heterogeneous in both composition and staging. The methods disclosed herein may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of a condition in a subject. Such methods can include, e.g., generating an aggregate profile of extracellular nucleic acids derived from the subject, wherein the aggregate profile comprises a plurality of data resulting from various nucleic acid analyses. In some embodiments, the aggregate profile comprises epigenetic and mutation analyses. In some embodiments, an aggregate profile comprises a summation of information derived from different cells in a heterogeneous disease. This summation may comprise structural variation identities and levels, copy number variation, epigenetic variation, or other mutation analyses.

The present methods can be used to diagnose, prognose, monitor or observe pre-cancers, cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### III. Additional Features of Certain Disclosed Methods

### A. Target Region Sets

In some embodiments, certain genomic regions of interest are detected and/or enriched. The genomic regions of interest may comprise one or more target region sets. In some embodiments, target region sets comprise variations that are not prevalent in DNA from healthy subjects or not prevalent in DNA obtained from healthy tissue regions. In some embodiments, target region sets comprise variations present in healthy cells but not normally present in the sample type, such as a blood sample. In some embodiments, the variations are present in aberrant cells (e.g., hyperplastic, metaplastic, or neoplastic cells). Exemplary target region sets include sequence-variable target region sets, epigenetic target region sets.

In some embodiments, a first target region set is detected, comprising at least epigenetic target regions. In some embodiments, the epigenetic target regions detected in a first subsample comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions show type-specific hypermethylation in healthy cfDNA from one or more related cell or tissue types. Without wishing to be bound by any particular theory, the presence of cancer cells may increase the shedding of DNA into the bloodstream (e.g., from the cancer and/or the surrounding tissue). As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, the methods herein comprise detecting a second captured target region set from a sample or second subsample, comprising at least epigenetic target regions. In some embodiments, the second epigenetic target region set comprises hypomethylation variable target regions. In some embodiments, the hypomethylation variable target regions are CpG-containing regions that are methylated or have high methylation in cfDNA from healthy subjects (e.g., above-average methylation relative to bulk cfDNA). Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

Additionally, target region sets may comprise DNA corresponding to a sequence-variable target region set.

### 1. Epigenetic Target Region Sets

In some embodiments, a target region set is or comprises an epigenetic target region set. Epigenetic target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation and Hypomethylation Variable Target Regions

In some embodiments, an epigenetic target region set comprises a hypermethylation variable target region. In some embodiments, the hypermethylation variable target regions are differentially or exclusively hypermethylated in one or more related cell or tissue types. Such hypermethylation variable target regions may be hypermethylated in other cell or tissue types but not to the extent observed in the one or more related cell or tissue types. In some embodiments, the hypermethylation variable target regions show even higher methylation in cfDNA from a diseased cell of the one or more related cell or tissue types.

In some embodiments, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In another example, as discussed above, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 1. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 1
Exemplary Hypermethylation Target
Regions based on CRC studies.

| | Additional | |
|---|---|---|
| Gene Name | Gene Name | Chromosome |
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, genomic regions targeted for sequencing comprise a plurality of loci listed in Table 1, e.g., at least 10%, 20%, 30%, 40%, 50%, 60%, 70%, 80%, 90%, or 100% of the loci listed in Table 1. In some embodiments, genomic regions are captured using probes. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5(5):492-504 (2016); Skvortsova et al., Br. J. Cancer. 94(10):1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, genomic regions targeted for sequencing comprise a plurality of loci listed in Table 1 or Table 2, e.g., at least 10%, 20%, 30%, 40%, 50%, 60%, 70%, 80%, 90%, or 100% of the loci listed in Table 1 or Table 2.

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, an epigenetic target region set comprises a hypomethylation variable target region. In some embodiments, the hypomethylation variable target regions are exclusively hypomethylated in one or more related cell or tissue types. Such hypomethylation variable target regions may be hypomethylated in other cell or tissue types but not to the extent observed in the one or more related cell or tissue types.

In some embodiments, where different epigenetic target regions are captured, the epigenetic target regions comprise hypermethylation and/or hypomethylation variable target regions.

Further exemplary hypermethylation variable target regions and hypomethylation variable target regions useful for distinguishing between various cell types have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol, 21, 156 (2020) (doi.org/10.1186/si3059-020-02065-5). Whole-genome bisulfite sequencing data is available from the Blueprint consortium, available on the internet at dcc.blueprint-epigenome.eu.

b. CTCF Binding Regions

In some embodiments, an epigenetic target region set comprises CTCF binding regions. CTCF is a DNA-binding protein that contributes to chromatin organization and often colocalizes with cohesin. Perturbation of CTCF binding sites has been reported in a variety of different cancers. See, e.g., Katainen et al., Nature Genetics, doi:10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Thus, perturbations of CTCF binding result in variation in the fragmentation patterns of cfDNA. As such, CTCF binding sites are a type of fragmentation variable target region.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

c. Transcription Start Sites

In some embodiments, an epigenetic target region set comprises variable transcrfiption start sites. Transcription start sites may show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to DNA that is typical in healthy subjects. Perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34(Database issue): D86-D89 (2006), which is incorporated herein by reference. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

d. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1.

e. Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### 2. Sequence-Variable Target Region Sets

In some embodiments, a target region set is or comprises a sequence-variable target region set. Sequence-variable target region sets may comprise one or more types of target regions likely to differentiate DNA from neoplastic (e.g., tumor or cancer) cells and from healthy cells, e.g., non-neoplastic circulating cells. Exemplary types of such regions are discussed in detail herein. The sequence-variable target region set may also comprise one or more control regions, e.g., as described herein. In some embodiments, a sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Examples of listings of genomic locations of interest may be found in, e.g., Table 3 and Table 4 herein. In some embodiments, a sequence-variable target region set comprises portions of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set comprises at least 5, at least 10, at least 15, at least 20, at least 25, at least 30, at least 35, at least 40, at least 45, at least 50, at least 55, at least 60, at least 65, or 70 of the SNVs of Table 3. In some embodiments, a sequence-variable target region set comprises portions of at least 1, at least 2, at least 3, at least 4, at least 5, or 6 of the fusions of Table 3. In some embodiments, a sequence-variable target region set comprises at least portions of at least 1, at least 2, or 3 of the indels of Table 3. In some embodiments, a sequence-variable target region set comprises portions of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, a sequence-variable target region set comprises at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, a sequence-variable target region set comprises portions of at least 1, at least 2, at least 3, at least 4, at least 5, or 6 of the fusions of Table 4. In some embodiments, a sequence-variable target region set comprises at least portions of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. Each of these genomic locations of interest may be identified as a backbone region or hot-spot region for a given panel. Table 5 shows an example listing of hot-spot genomic locations of interest. In some embodiments, a sequence-variable target region set comprises portions of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5. Each hot-spot genomic region is listed with the associated gene, chromosome on which it resides, the start and stop position of the genome representing the gene's locus, the length of the gene's locus in base pairs, the exons covered by the gene, and the critical feature (e.g., type of mutation) of a given genomic region of interest.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 67 | promoter | chr5:1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 (total | | |
| | | | | target | | |
| | | | | region) | | |
| | | | | 16330 (total | | |
| | | | | probe | | |
| | | | | coverage) | | |

Examples of listings of target regions of interest may also be found in WO 2020/160414, e.g., at Table 4. Additional examples include loci disclosed in Gale et al., PLoS One 13: e0194630 (2018), incorporated herein by reference, which describes a panel of 35 cancer-related gene targets: AKT1, ALK, BRAF, CCND1, CDK2A, CTNNB1, EGFR, ERB1B2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1. In some embodiments, the sequence-variable target region set comprises target regions from at least 10, 20, 30, or 35 cancer-related genes, such as the cancer-related genes listed herein and in WO 2020/160414.

In some embodiments, the sequence-variable target region set has a footprint of at least 50 kbp, e.g., at least 100 kbp, at least 200 kbp, at least 300 kbp, or at least 400 kbp. In some embodiments, the sequence-variable target region set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

### B. Collections of Target-Specific Probes

In some embodiments, a collection of target-specific probes is used in methods described herein. In some embodiments, the collection of target-specific probes comprises target-binding probes specific for a sequence-variable target region set and target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39: 8740-8751 (2011); Freier et al., Nucleic Acids Res. 25: 4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set are a bait set as discussed above, e.g., probes comprising capture moieties and sequences selected to tile across a panel of regions, such as genes.

In some embodiments, the target-specific probes are provided in a single composition. The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### C. Computer Systems

Methods of the present disclosure can be implemented using, or with the aid of, computer systems. For example, such methods may comprise: performing a methylation-preserving amplification of the DNA, wherein the DNA comprises barcodes; sequencing the DNA in a modification-sensitive manner; and determining an epigenetic consensus sequence of the DNA associated with at least a portion of the barcodes. By way of another example, such methods may comprise: performing a methylation-preserving amplification of the DNA, wherein the DNA comprises barcodes; subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and sequencing the DNA and determining an epigenetic consensus sequence of the DNA associated with at least a portion of the barcodes. In another example, such methods may comprise: performing a methylation-preserving amplification of the DNA, wherein the DNA comprises barcodes; enriching for one or more sets of epigenetic target regions of DNA from the DNA, thereby providing enriched DNA; sequencing the enriched DNA in a modification-sensitive manner and determining an epigenetic consensus sequence of the enriched DNA associated with at least a portion of the barcodes. Exemplary methods may comprise linear methylation-preserving amplification, wherein barcoding is optional.

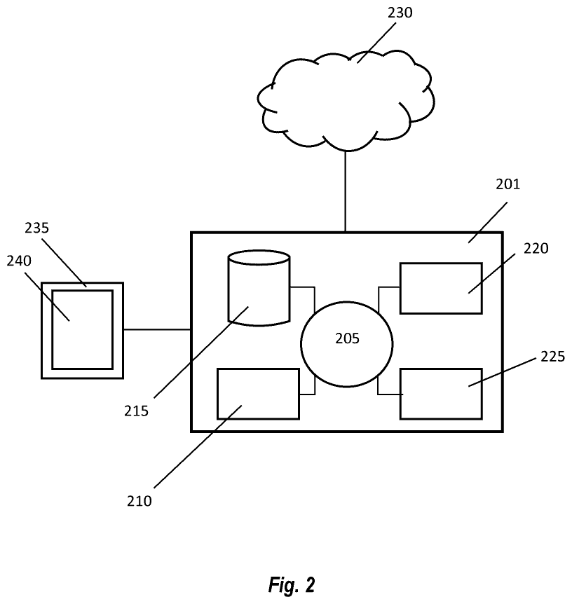

FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 0, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method comprising: collecting DNA from a test subject; performing a methylation-preserving amplification of the DNA, wherein the DNA comprises barcodes; sequencing the DNA in a modification-sensitive manner; and determining an epigenetic consensus sequence of the DNA associated with at least a portion of the barcodes. In another aspect, the present disclosure provides a non-transitory computer-readable medium comprising computer-executable instructions which, when executed by at least one electronic processor, perform at least a portion of a method comprising: collecting DNA from a test subject; performing a methylation-preserving amplification of the DNA, wherein the DNA comprises barcodes; subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and sequencing the DNA and determining an epigenetic consensus sequence of the DNA associated with at least a portion of the barcodes. In yet another aspect, the present disclosure provides a non-transitory computer-readable medium comprising computer-executable instructions which, when executed by at least one electronic processor, perform at least a portion of a method comprising: collecting DNA from a test subject; performing a methylation-preserving amplification of the DNA, wherein the DNA comprises barcodes; enriching for one or more sets of epigenetic target regions of DNA from the DNA, thereby providing enriched DNA; sequencing the enriched DNA in a modification-sensitive manner and determining an epigenetic consensus sequence of the enriched DNA associated with at least a portion of the barcodes. Exemplary methods may comprise linear methylation-preserving amplification, wherein barcoding is optional.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud ComputingArchitected: Solution Design Handbook, Recursive Press (2011), each of which is hereby incorporated by reference in its entirety.

### D. Applications

### 1. Cancer and Other Diseases; Cell Type Quantification

The present methods can be used to diagnose presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may result in changes in levels of different immune cell types (including rare immune cell types), and/or increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a leukapheresis sample, or a PBMC sample) from the subject) if the treatment is successful as more cancer cells may die and shed DNA, or, e.g., if a successful treatment results in an increase or decrease in the quantity of a specific protein in the blood and an unsuccessful treatment results in no change. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor the likelihood of residual disease or the likelihood of recurrence of disease.

In some embodiments, the present methods are used for screening for a cancer, or in a method for screening cancer. For example, the sample can be a sample from a subject who has not been previously diagnosed with cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at www.dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast cancer, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolernia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules. Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardio facial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

Furthermore, in some embodiments, the one or more methods described in the present disclosure may be used to assist in the treatment of a type of cancer.

These methods provided herein provide a deeper understanding of the changes in DNA and proteins that cause cancer, allowing the identification of biomarkers and design of treatments that target these proteins. In some embodiments, the biomarker may include an epigenetic signature, such as a methylation state, methylation score and/or DNA fragmentation pattern/score. In some embodiments, the epigenetic signature can be determined for one or more regions that include, but not limited to, transcription start sites, promoter regions, CTCF binding regions and regulatory protein binding regions. In some embodiments, the epigenetic signature is determined for one or more regions that include, but not limited to, transcription start sites, promoter regions, intergenic regions and/or intronic regions that are associated with at least one or more genes listed in any one or more of Tables 1, 2, 3, 4, and 5. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells.

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Subject and/or Classifying a Subject as being a Candidate for a Subsequent Cancer Treatment

In some embodiments, a method provided herein is a method of determining a risk of cancer recurrence in a subject. In some embodiments, a method provided herein is a method of classifying a subject as being a candidate for a subsequent cancer treatment.

Any of such methods may comprise collecting DNA (e.g., originating or derived from a tumor cell) from the subject diagnosed with the cancer at one or more preselected timepoints following one or more previous cancer treatments to the subject. The subject may be any of the subjects described herein. The DNA may be DNA, such as cfDNA, from a blood sample (e.g., a whole blood sample, a buffy coat sample, a leukapheresis sample, or a PBMC sample). The DNA may comprise DNA obtained from a tissue sample.

Any of such methods may comprise capturing a plurality of sets of target regions from DNA from the subject, wherein the plurality of target region sets comprise a sequence-variable target region set, and/or an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of a sequence-variable target region set may be sequenced to a greater depth of sequencing than the captured DNA molecules of the epigenetic target region set.

Any of such methods may comprise detecting a presence or absence of DNA, such as cfDNA, originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as cfDNA, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, from a blood sample (e.g., a whole blood sample, a buffy coat sample, a leukapheresis sample, or a PBMC sample), and/or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening), such as based on changes in levels of different immune cell types, including rare immune cell types, in samples collected from a subject over time. In some embodiments, monitoring comprises analysis of at least two samples collected from a subject at at least two different time points as described herein.

The methods according to the present disclosure can also be useful in predicting a subject's response to a particular treatment option. Successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur. In another example, certain treatment options may be correlated with profiles (e.g., of immune cell types, proteins, and/or genetic profiles) of cancers over time. This correlation may be useful in selecting a therapy for a subject.

The disclosed methods can include evaluating (such as quantifying) and/or interpreting a quantity of one or more different immune cell types (including rare immune cell types), of a DNA sequence (such as one or more CDR3 sequences), and/or a protein or proteins present in one or more samples, such as one or more samples comprising cells or a blood sample (e.g., a buffy coat sample, a whole blood sample, a leukapheresis sample, or a PBMC sample), collected from a subject at one or more timepoints in comparison to a selected baseline value or reference standard (or a selected set of baseline values or reference standards). A baseline value or reference standard may be a quantity of the one or more different immune cell types (including rare immune cell types), a quantity of a DNA sequence (such as one or more CDR3 sequences), and/or a quantity of the protein or proteins measured in one or more samples (such as an average quantity or range of quantities of the protein or proteins present in at least two samples) collected from the subject at one or more time points, such as prior to receiving a treatment, prior to diagnosis of a condition (such as a cancer), or as part of a preventative health monitoring program. A baseline value or reference standard may be a quantity of the one or more different immune cell types (including rare immune cell types), a quantity of a DNA sequence (such as one or more CDR3 sequences), and/or a quantity of the protein or proteins measured in one or more samples (such as an average quantity or range of quantities of the protein or proteins present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a sample comprising cells or a blood sample (e.g., a buffy coat sample, a whole blood sample, a leukapheresis sample, or a PBMC sample) may be collected from a subject at two or more timepoints, to assess changes in the quantity of one or more immune cell types, the quantity of one or more DNA sequences (e.g., one or more CDR3 sequences), or the quantity of a protein or proteins (such as changes in quantities of the protein or proteins, or changes in one or more modifications (such as one or more post-translational modifications) of the protein or proteins) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring the quantity of one or more immune cell types, the quantity of one or more DNA sequences (e.g., one or more CDR3 sequences), or the quantity a protein or proteins and identifying differences between the immune cell types, DNA sequences, and/or protein or proteins in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein the quantity of one or more immune cell types, the quantity of one or more DNA sequences (e.g., one or more CDR3 sequences), or the quantity of a protein or proteins present in at least one sample (such as at least one whole blood sample, buffy coat sample, leukapheresis sample, or PBMC sample) collected from a subject at one or more timepoints (such as prior to receiving a treatment) is compared to the quantity of the one or more immune cell types, the quantity of the one or more DNA sequences (e.g., one or more CDR3 sequences), or the quantity of the protein or proteins present in at least one sample collected from the subject at one or more different time points (such as after receiving the treatment). The disclosed methods can allow for patient-specific monitoring, such that, for example, differences in immune cell type quantities, DNA sequence quantities (such as CDR3 sequence quantities, such as quantities of T-cell or B-cell specific CDR3 sequences), protein quantities and/or protein modifications between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples, such as a sample comprising cells or a blood sample (such as one or more whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples, such one or more samples comprising cells or one or more blood samples, e.g., one or more buffy coat samples, whole blood samples, leukapheresis samples, or PBMC samples, are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to patients or subjects. In some embodiments, determination of the levels of particular immune cell types, including rare immune cell types, facilitates selection of appropriate treatment. In some embodiments, the patient or subject has a given disease, disorder or condition. In some embodiments of the disclosed methods, wherein the DNA and/or the sample is from a subject, the method further comprises determining the presence or status of a cancer in the subject. In some embodiments, determining the presence or status of a cancer in the subject can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject, e.g., at one or more timepoints, in comparison to a selected baseline value or reference standard (or a selected set of baseline values or reference standards), such as described in further detail below. In some such embodiments, the method further comprises administering at least one cancer therapy to a subject in need thereof (e.g., based on a determination of presence of a cancer in a subject).

Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

Therapeutic agents such as immunotherapeutic agents can function by helping the immune system destroy cancer cells. For example, certain targeted therapeutic agents may mark cancer cells for the immune system to destroy them. Other targeted therapeutic agents may support the immune system to work more effectively against cancer. Yet other therapeutic agents may stop cancer cells from growing, for example, by interfering with cancer cell surface markers preventing them from dividing. Additionally, therapeutic agents can inhibit signals that promote angiogenesis. Such angiogenesis inhibitors prevent blood supply into the tumor thereby, preventing tumor growth. Other targeted therapeutic agents can deliver toxic substances to the tumor. Examples include monoclonal antibodies combined with toxins, chemotherapy, or radiation. Some targeted therapeutic agents induce apoptosis or deplete cancer of hormones.

In some embodiments, the therapeutic agents comprise one or more of PARP inhibitors, such as Olaparib (Lynparza), Rucaparib (Rubraca), Niraparib (Zejula), and Talazoparib (Talzenna). These may be used for treating mutations in BRCA1, BRCA2, ATM, BARD1, BRIP1, CDK12, CHEK1, CHEK2, FANCL, PALB2, RAD51B, RAD51 C, RAD51D and RAD54L alterations, and/or for genes associated Homologous Recombination Repair (HRR).

In some embodiments the treatment comprises one or more immunotherapies, immunotherapeutic agents and/or immune checkpoint inhibitors (ICIS). Immunotherapies are treatments with one or more agents that act to stimulate the immune system so as to kill or at least to inhibit growth of cancer cells, and preferably to reduce further growth of the cancer, reduce the size of the cancer and/or eliminate the cancer. Some such agents bind to a target present on cancer cells; some bind to a target present on immune cells and not on cancer cells; some bind to a target present on both cancer cells and immune cells. Such agents include, but are not limited to, checkpoint inhibitors and/or antibodies. Checkpoint inhibitors are inhibitors of pathways of the immune system that maintain self-tolerance and modulate the duration and amplitude of physiological immune responses in peripheral tissues to minimize collateral tissue damage (see, e.g., Pardoll, Nature Reviews Cancer 12, 252-264 (2012)). Exemplary agents include antibodies against any of PD-1, PD-2, PD-L1, PD-L2, CTLA-4, OX40, B7.1, B7He, LAG3, CD137, KIR, CCR5, CD27, CD40, or CD47. Other exemplary agents include proinflammatory cytokines, such as IL-1, IL-6, and TNF-α. Other exemplary agents are T-cells activated against a tumor, such as T-cells activated by expressing a chimeric antigen targeting a tumor antigen recognized by the T-cell. In some embodiments, anti-PD-1 or anti-PD-L1 therapies comprise pembrolizumab (Keytruda), nivolumab (Opdivo), and cemiplimab (Libtayo), atezolizumab (Tecentriq), durvalumab (Imfinzi), and avelumab (Bavencio). These therapies may be used to treat patients identified as having high microsatellite instability (MSI) status or high tumor mutational burden (TMB).

In some embodiments, a therapeutic agent targets a mutated form of the EGFR protein. Such therapeutic agents can include osimertinib (Tagrisso), erlotinib (Tarceva), and gefinitib (Iressa).

Therapeutic agents can include one or more targeted therapeutic agents, including any one or more of abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aligopa), crizotinib (Xalkori), dabrafenib (Tafmlar), dabrafenib mesylate (Tafmlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane 1 131 (Azedra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk(Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride(Votrient), pembrolizumab (Keytruda), pemigatinib(Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate(Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

Table 6 provides an exemplary list of drugs used to treat mutations observed in target genes associated with certain cancer types.

TABLE 6
Exemplary drugs

| Cancer type | Drug | Target genes |
|---|---|---|
| Breast | abemaciclib (Verzenio) | CDK4, CDK6 |
| prostate | abiraterone acetate (Zytiga) | CYP17A1 |
| leukemia | acalabrutinib (Calquence) | BTK |
| lymphoma | acalabrutinib (Calquence) | BTK |
| lung | adagrasib (Krazati) | KRAS G12C |
| Breast | ado-trastuzumab emtansine | Tubulin, Her2 |
| | (Kadcyla) | |
| lung | afatinib dimaleate (Gilotrif) | EGFR + EGFR exon 21 L858R |
| | | mutation + EGFR-Ex19del + |
| | | HER2 + HER4 |
| lung | alectinib (Alecensa) | ALK, RET |
| leukemia | alemtuzumab (Campath) | CD52 |
| skin | alitretinoin (Panretin) | RARα + RARβ2 + RARγ |
| soft tissue | alitretinoin (Panretin) | RARα + RARβ2 + RARγ |
| sarcoma | | |
| Breast | alpelisib (Piqray) | PI3Kα |
| lung | amivantamab-vmjw | EGFR + c-MET |
| | (Rybrevant) | |
| Breast | anastrozole (Arimidex) | Aromatase |
| prostate | apalutamide (Erleada) | AR |
| leukemia | asciminib hydrochloride | Bcr-Abl |
| | (Scemblix) | |
| bladder | atezolizumab (Tecentriq) | PDL1 |
| liver and bile | atezolizumab (Tecentriq) | PDL1 |
| duct | | |
| lung | atezolizumab (Tecentriq) | PDL1 |
| skin | atezolizumab (Tecentriq) | PDL1 |
| soft tissue | atezolizumab (Tecentriq) | PDL1 |
| sarcoma | | |
| Gastrointestinal | avapritinib (Ayvakit) | PDGFRα + c-Kit |
| systemic | avapritinib (Ayvakit) | PDGFRα + c-Kit |
| mastocytosis | | |
| bladder | avelumab (Bavencio) | PDL1 |
| endocrine | avelumab (Bavencio) | PDL1 |
| and | | |
| neuroendocrine | | |
| tumors | | |
| kidney | avelumab (Bavencio) | PDL1 |
| skin | avelumab (Bavencio) | PDL1 |
| lymphoma | axicabtagene ciloleucel | CD19 |
| | (Yescarta) | |
| kidney | axitinib (Inlyta) | VEGFR1 + VEGFR2 + VEGFR3 |
| lymphoma | belinostat (Beleodaq) | HDAC |
| Brain | belzutifan (Welireg) | HIF-2A |
| kidney | belzutifan (Welireg) | HIF-2A |
| pancreatic | belzutifan (Welireg) | HIF-2A |
| ovarian | bevacizumab (Avastin) | VEGF-A |
| epithelial, | | |
| fallopian | | |
| tube, and | | |
| primary | | |
| peritoneal | | |
| Brain | bevacizumab (Avastin) | VEGF-A |
| Cervical | bevacizumab (Avastin) | VEGF-A |
| colorectal | bevacizumab (Avastin) | VEGF-A |
| kidney | bevacizumab (Avastin) | VEGF-A |
| liver and bile | bevacizumab (Avastin) | VEGF-A |
| duct | | |
| lung | bevacizumab (Avastin) | VEGF-A |
| lymphoma | bexarotene (Targretin) | RXRs |
| skin | binimetinib (Mektovi) | MEK1, MEK2 |
| leukemia | blinatumomab (Blincyto) | CD19, CD3 |
| lymphoma | bortezomib (Velcade) | Proteasome |
| multiple | bortezomib (Velcade) | Proteasome |
| myeloma | | |
| leukemia | bosutinib (Bosulif) | BCR-ABL, SRC |
| lymphoma | brentuximab vedotin | CD130, Tubulin |
| | (Adcetris) | |
| leukemia | brexucabtagene autoleucel | CD19 |
| | (Tecartus) | |
| lymphoma | brexucabtagene autoleucel | CD19 |
| | (Tecartus) | |
| lung | brigatinib (Alunbrig) | ALK, EGFR , FLT3, IGF-IR, |
| | | ROS1 |
| prostate | cabazitaxel (Jevtana) | Tubulin |
| kidney | cabozantinib-s-malate | AXL + RET + ROS1 + TYRO3 + |
| | (Cabometyx) | Tie-2 + TrkB + VEGFR1 + |
| | | VEGFR2 + VEGFR3 + c-Kit + c- |
| | | Met |
| liver and bile | cabozantinib-s-malate | AXL + RET + ROS1 + TYRO3 + |
| duct | (Cabometyx) | Tie-2 + TrkB + VEGFR1 + |
| | | VEGFR2 + VEGFR3 + c-Kit + c- |
| | | Met |
| thyroid | cabozantinib-s-malate | AXL + RET + ROSI + TYRO3 + |
| | (Cometriq) | Tie-2 + TrkB + VEGFR1 + |
| | | VEGFR2 + VEGFR3 + c-Kit + c- |
| | | Met |
| lung | capmatinib hydrochloride | c-Met |
| | (Tabrecta) | |
| multiple | carfilzomib (Kyprolis) | protoasome |
| myeloma | | |
| lung | cemiplimab-rwlc (Libtayo) | PD-1 |
| skin | cemiplimab-rwlc (Libtayo) | PD-1 |
| lung | ceritinib (Zykadia) | ALK + IGF-1R + INSR + ROS1 |
| colorectal | cetuximab (Erbitux) | EGFR |
| multiple | ciltacabtagene autoleucel | BCMA |
| myeloma | (Carvykti) | |
| skin | cobimetinib fumarate | MEK1, MEK2 |
| | (Cotellic) | |
| lymphoma | copanlisib hydrochloride | PI3Kα , PI3Kδ |
| | (Aliqopa) | |
| lung | crizotinib (Xalkori) | MTH1 |
| lymphoma | crizotinib (Xalkori) | MTH1 |
| Myofibroblastic | crizotinib (Xalkori) | MTH1 |
| Brain | dabrafenib (Tafinlar) | BRAF, CRAF |
| lung | dabrafenib mesylate | BRAF, CRAF |
| | (Tafinlar) | |
| skin | dabrafenib mesylate | BRAF, CRAF |
| | (Tafinlar) | |
| solid tumors | dabrafenib mesylate | BRAF, CRAF |
| anywhere in | (Tafinlar) | |
| the body | | |
| thyroid | dabrafenib mesylate | BRAF, CRAF |
| | (Tafinlar) | |
| lung | dacomitinib (Vizimpro) | EGFR + EGFR exon 21 L858R |
| | | mutation + EGFR-Ex19del + |
| | | HER2 + HER4 |
| multiple | daratumumab (Darzalex) | CD38 + Hyaluronic acid |
| myeloma | | |
| multiple | daratumumab and | CD38 + Hyaluronic acid |
| myeloma | hyaluronidase-fihj (Darzalex | |
| | Faspro) | |
| prostate | darolutamide (Nubeqa) | AR |
| leukemia | dasatinib (Sprycel) | Bcr-Abl + EphA2 + FYN + LCK + |
| | | PDGFRβ + Protein-tyrosine |
| | | kinases + SRC + YES1 + c-Kit |
| neuroblastoma | dinutuximab (Unituxin) | GD2 |
| endometrial | dostarlimab-gxly (Jemperli) | PD-1 |
| solid tumors | dostarlimab-gxly (Jemperli) | PD-1 |
| anywhere in | | |
| the body | | |
| liver and bile | durvalumab (Imfinzi) | PDL1 |
| duct | | |
| lung | durvalumab (Imfinzi) | PDL1 |
| leukemia | duvelisib (Copiktra) | PI3Kγ + PI3Kδ |
| Breast | elacestrant dihydrochloride | Erα/ESR1 |
| | (Orserdu) | |
| multiple | elotuzumab (Empliciti) | SLAMF7 |
| myeloma | | |
| leukemia | enasidenib mesylate (Idhifa) | IDH2 |
| colorectal | encorafenib (Braftovi) | BRAF ,BRAF V600E |
| skin | encorafenib (Braftovi) | BRAF V600E , V600K mutation |
| bladder | enfortumab vedotin-ejfv | Nectin-4, Tubulins |
| | (Padcev) | |
| lung | entrectinib (Rozlytrek) | ALK + ROS1 + TRKA + TrkB + |
| | | TrkC |
| solid tumors | entrectinib (Rozlytrek) | ALK + ROS1 + TRKA + TrkB + |
| anywhere in | | TrkC |
| the body | | |
| prostate | enzalutamide (Xtandi) | AR |
| bladder | erdafitinib (Balversa) | FGFR1, FGFR2, FGFR3, FGFR4 |
| lung | erlotinib hydrochloride | EGFR antagonists , EGFR exon |
| | (Tarceva) | 21 L858R mutation inhibitors , |
| | | EGFR-Ex19del inhibitors |
| pancreatic | erlotinib hydrochloride | EGFR + EGFR exon 21 L858R |
| | (Tarceva) | mutation + EGFR-Ex19del |
| Breast | everolimus (Afinitor) | mTORC1, mTORC2 |
| kidney | everolimus (Afinitor) | mTORC1, mTORC2 |
| pancreatic | everolimus (Afinitor) | mTORC1, mTORC2 |
| Breast | exemestane (Aromasin) | Aromatase |
| Breast | fam-trastuzumab | HER2, TOP1 |
| | deruxtecan-nxki (Enhertu) | |
| Gastric | fam-trastuzumab | HER2, TOP1 |
| | deruxtecan-nxki (Enhertu) | |
| lung | fam-trastuzumab | HER2, TOP1 |
| | deruxtecan-nxki (Enhertu) | |
| myelodysplastic | fedratinib hydrochloride | JAK2, FLT3 |
| and | (Inrebic) | |
| myeloproliferative | | |
| disorders | | |
| Breast | fulvestrant (Faslodex) | ER |
| liver and bile | futibatinib (Lytgobi) | FGFRs |
| duct | | |
| lung | gefitinib (Iressa) | EGFR + EGFR exon 21 L858R |
| | | mutation + EGFR-Ex19del |
| leukemia | gemtuzumab ozogamicin | CD33, DNA |
| | (MYLOTARG) | |
| leukemia | gilteritinib fumarate | AXL + FLT3 |
| | (Xospata) | |
| leukemia | glasdegib | SMO |
| | maleate (Daurismo) | |
| lymphoma | ibritumomab tiuxetan | CD20 |
| | (Zevalin) | |
| leukemia | ibrutinib (Imbruvica) | BTK |
| lymphoma | ibrutinib (Imbruvica) | BTK |
| multiple | idecabtagene vicleucel | BCMA |
| myeloma | (Abecma) | |
| leukemia | idelalisib (Zydelig) | PI3Kδ |
| dermatofibro- | imatinib mesylate (Gleevec) | BCR-ABL, PDGFR, C-Kit |
| sarcoma | | |
| protuberans | | |
| Gastrointestinal | imatinib mesylate (Gleevec) | BCR-ABL, PDGFR, C-Kit |
| leukemia | imatinib mesylate (Gleevec) | BCR-ABL, PDGFR, C-Kit |
| myelodysplastic | imatinib mesylate (Gleevec) | BCR-ABL, PDGFR, C-Kit |
| and | | |
| myeloproliferative | | |
| disorders | | |
| systemic | imatinib mesylate (Gleevec) | BCR-ABL, PDGFR, C-Kit |
| mastocytosis | | |
| liver and bile | infigratinib phosphate | FGFR1 + FGFR2 + FGFR3 + |
| duct | (Truseltiq) | FGFR4 |
| leukemia | inotuzumab ozogamicin | CD22 + DNA |
| | (Besponsa) | |
| endocrine | iobenguane I 131 (Azedra) | NET |
| and | | |
| neuroendocrine | | |
| tumors | | |
| colorectal | ipilimumab (Yervoy) | CTLA4 |
| esophageal | ipilimumab (Yervoy) | CTLA4 |
| kidney | ipilimumab (Yervoy) | CTLA4 |
| liver and bile | ipilimumab (Yervoy) | CTLA4 |
| duct | | |
| lung | ipilimumab (Yervoy) | CTLA4 |
| malignant | ipilimumab (Yervoy) | CTLA4 |
| mesothelioma | | |
| skin | ipilimumab (Yervoy) | CTLA4 |
| multiple | isatuximab-irfc (Sarclisa) | CD38 |
| myeloma | | |
| leukemia | ivosidenib (Tibsovo) | IDH1 |
| multiple | ixazomib citrate (Ninlaro) | proteasome |
| myeloma | | |
| endocrine | lanreotide acetate | SSTR |
| and | (Somatuline Depot) | |
| neuroendocrine | | |
| tumors | | |
| Breast | lapatinib ditosylate (Tykerb) | EGFR |
| solid tumors | larotrectinib sulfate | TRKA + TrkB + TrkC |
| anywhere in | (Vitrakvi) | |
| the body | | |
| endometrial | lenvatinib mesylate | FGFR1 + FGFR2 + FGFR3 + |
| | (Lenvima) | FGFR4 + PDGFRα + RET + |
| | | VEGFR1 + VEGFR2 + |
| | | VEGFR3 + c-Kit |
| kidney | lenvatinib mesylate | FGFR1 + FGFR2 + FGFR3 + |
| | (Lenvima) | FGFR4 + PDGFRα + RET + |
| | | VEGFR1 + VEGFR2 + |
| | | VEGFR3 + c-Kit |
| liver and bile | lenvatinib mesylate | FGFR1 + FGFR2 + FGFR3 + |
| duct | (Lenvima) | FGFR4 + PDGFRα + RET + |
| | | VEGFR1 + VEGFR2 + |
| | | VEGFR3 + c-Kit |
| thyroid | lenvatinib mesylate | FGFR1 + FGFR2 + FGFR3 + |
| | (Lenvima) | FGFR4 + PDGFRα + RET + |
| | | VEGFR1 + VEGFR2 + |
| | | VEGFR3 + c-Kit |
| Breast | letrozole | CDK4/ER |
| | (Femara)/Ribociclib | |
| | Succinate | |
| lymphoma | lisocabtagene maraleucel | CD19 |
| | (Breyanzi) | |
| lymphoma | loncastuximab tesirine-lpyl | CD19 + DNA |
| | (Zynlonta) | |
| lung | lorlatinib (Lorbrena) | ALK + ROS1 |
| prostate | lutetium Lu 177 vipivotide | PSMA |
| | tetraxetan (Pluvicto) | |
| endocrine | lutetium Lu 177-dotatate | SSTR2 |
| and | (Lutathera) | |
| neuroendocrine | | |
| tumors | | |
| Breast | margetuximab-cmkb | HER2 |
| | (Margenza) | |
| leukemia | midostaurin (Rydapt) | FLT3 + PDGFR + PKC + Syk + |
| | | VEGFR2 + c-Kit |
| systemic | midostaurin (Rydapt) | FLT3 + PDGFR + PKC + Syk + |
| mastocytosis | | VEGFR2 + c-Kit |
| ovarian | mirvetuximab soravtansine- | FOLR1 + Tubulin |
| epithelial, | gynx (Elahere) | |
| fallopian | | |
| tube, and | | |
| primary | | |
| peritoneal | | |
| lung | mobocertinib succinate | EGFR exon 20 + HER2 exon 20 |
| | (Exkivity) | |
| lymphoma | mogamulizumab-kpkc | CCR4 |
| | (Poteligeo) | |
| lymphoma | mosunetuzumab-axgb | CD20, CD3 |
| | (Lunsumio) | |
| leukemia | moxetumomab pasudotox- | CD22 |
| | tdfk (Lumoxiti) | |
| neuroblastoma | naxitamab-gqgk (Danyelza) | GD2 |
| lung | necitumumab (Portrazza) | EGFR |
| Breast | neratinib maleate (Nerlynx) | EGFR, HER2, HER4 |
| leukemia | nilotinib (Tasigna) | Bcr-Abl + CSF-1R + DDR1 + |
| | | PDGFR + c-Kit |
| ovarian | niraparib tosylate | PARP1 + PARP2 |
| epithelial, | monohydrate (Zejula) | |
| fallopian | | |
| tube, and | | |
| primary | | |
| peritoneal | | |
| bladder | nivolumab (Opdivo) | PD-1 |
| colorectal | nivolumab (Opdivo) | PD-1 |
| esophageal | nivolumab (Opdivo) | PD-1 |
| kidney | nivolumab (Opdivo) | PD-1 |
| liver and bile | nivolumab (Opdivo) | PD-1 |
| duct | | |
| lung | nivolumab (Opdivo) | PD-1 |
| lymphoma | nivolumab (Opdivo) | PD-1 |
| malignant | nivolumab (Opdivo) | PD-1 |
| mesothelioma | | |
| skin | nivolumab (Opdivo) | PD-1 |
| stomach | nivolumab (Opdivo) | PD-1 |
| (gastric) | | |
| skin | nivolumab and relatlimab- | LAG3 + PD-1 |
| | rmbw (Opdualag) | |
| leukemia | obinutuzumab (Gazyva) | CD20 |
| lymphoma | obinutuzumab (Gazyva) | CD20 |
| leukemia | ofatumumab (Arzerra) | CD20 |
| ovarian | olaparib (Lynparza) | PARP1, PARP2, PARP3 |
| epithelial, | | |
| fallopian | | |
| tube, and | | |
| primary | | |
| peritoneal | | |
| Breast | olaparib (Lynparza) | PARP1, PARP2, PARP3 |
| pancreatic | olaparib (Lynparza) | PARP1, PARP2, PARP3 |
| prostate | olaparib (Lynparza) | PARP1, PARP2, PARP3 |
| leukemia | olutasidenib (Rezlidhia) | IDH1 |
| lung | osimertinib mesylate | EGFR + EGFR T790M + EGFR |
| | (Tagrisso) | exon 21 L858R mutation + |
| | | EGFR-Ex19del |
| myelodysplastic | pacritinib citrate (Vonjo) | CSF-1R + FLT3 + IRAK1 + |
| and | | JAK2 |
| myeloproliferative | | |
| disorders | | |
| Breast | palbociclib (Ibrance) | CDK4, CDK6 |
| colorectal | panitumumab (Vectibix) | EGFR |
| kidney | pazopanib | FGFR1 + FGFR3 + Flt3L + |
| | hydrochloride (Votrient) | ITK + LCK + PDGFRα + |
| | | PDGFRB + VEGFR1 + |
| | | VEGFR2 + VEGFR3 + c-Kit |
| soft tissue | pazopanib hydrochloride | FGFR1 + FGFR3 + Flt3L + |
| sarcoma | (Votrient) | ITK + LCK + PDGFRα + |
| | | PDGFRB + VEGFR1 + VEGFR2 + |
| | | VEGFR3 + c-Kit |
| Breast | pembrolizumab (Keytruda) | PD-1 |
| Cervical | pembrolizumab (Keytruda) | PD-1 |
| colorectal | pembrolizumab (Keytruda) | PD-1 |
| endometrial | pembrolizumab (Keytruda) | PD-1 |
| esophageal | pembrolizumab (Keytruda) | PD-1 |
| kidney | pembrolizumab (Keytruda) | PD-1 |
| liver and bile | pembrolizumab (Keytruda) | PD-1 |
| duct | | |
| lung | pembrolizumab (Keytruda) | PD-1 |
| lymphoma | pembrolizumab (Keytruda) | PD-1 |
| skin | pembrolizumab (Keytruda) | PD-1 |
| solid tumors | pembrolizumab (Keytruda) | PD-1 |
| anywhere in | | |
| the body | | |
| stomach | pembrolizumab (Keytruda) | PD-1 |
| (gastric) | | |
| leukemia | pemigatinib (Pemazyre) | FGFR1, FGFR2, FGFR3, FGFR4 |
| liver and bile | pemigatinib (Pemazyre) | FGFR1, FGFR2, FGFR3, FGFR4 |
| duct | | |
| lymphoma | pemigatinib (Pemazyre) | FGFR1, FGFR2, FGFR3, FGFR4 |
| myelodysplastic | pemigatinib (Pemazyre) | FGFR1, FGFR2, FGFR3, FGFR4 |
| and | | |
| myeloproliferative | | |
| disorders | | |
| Breast | pertuzumab (Perjeta) | HER2 |
| Breast | pertuzumab, trastuzumab, | HER2, Hyaluronic acid |
| | and hyaluronidase-zzxf | |
| | (Phesgo) | |
| giant cell | pexidartinib hydrochloride | CSF-1R + FLT3 + c-Kit |
| tumor | (Turalio) | |
| lymphoma | pirtobrutinib (Jaypirca) | BTK C481S |
| lymphoma | polatuzumab vedotin-piiq | CD79B + Tubulin |
| | (Polivy) | |
| leukemia | ponatinib hydrochloride | Bcr-Abl + Protein-tyrosine |
| | (Iclusig) | kinases |
| lymphoma | pralatrexate (Folotyn) | Bcr-Abl + Protein-tyrosine |
| | | kinases |
| lung | pralsetinib (Gavreto) | RET |
| thyroid | pralsetinib (Gavreto) | RET |
| prostate | radium 223 dichloride | DNA |
| | (Xofigo) | |
| colorectal | ramucirumab (Cyramza) | VEGFR2 |
| Gastric | ramucirumab (Cyramza) | VEGFR2 |
| liver and bile | ramucirumab (Cyramza) | VEGFR2 |
| duct | | |
| lung | ramucirumab (Cyramza) | VEGFR2 |
| stomach | ramucirumab (Cyramza) | VEGFR2 |
| (gastric) | | |
| colorectal | regorafenib (Stivarga) | Abl family, BRAF, BRAF |
| | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| Gastrointestinal | regorafenib (Stivarga) | Abl family, BRAF, BRAF |
| | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| liver and bile | regorafenib (Stivarga) | Abl family, BRAF, BRAF |
| duct | | V600E, CRAF, CSF-1R, DDR2, |
| | | EphA2, FGFR1, FGFR2, FRK, |
| | | MAPK11, PDGFRa, PDGFRb, |
| | | RET, TRKA, Tie-2, VEGFR1, |
| | | VEGFR2, VEGFR3, c-kit |
| skin | retifanlimab-dlwr (Zynyz) | PD-1 |
| Breast | ribociclib (Kisqali) | CDK4, CDK6 |
| gastrointestinal | ripretinib (Qinlock) | EGFR + PDGFRα + c-Kit |
| leukemia | rituximab (Rituxan) | CD20 + Hyaluronic acid |
| lymphoma | rituximab (Rituxan) | CD20 + Hyaluronic acid |
| leukemia | rituximab and hyaluronidase | CD20 + Hyaluronic acid |
| | human (Rituxan Hycela) | |
| lymphoma | rituximab and hyaluronidase | CD20 + Hyaluronic acid |
| | human (Rituxan Hycela) | |
| lymphoma | romidepsin (Istodax) | HDAC |
| ovarian | rucaparib | PARP1 + PARP2 + PARP3 |
| epithelial, | camsylate (Rubraca) | |
| fallopian | | |
| tube, and | | |
| primary | | |
| peritoneal | | |
| prostate | rucaparib camsylate | PARP1 + PARP2 + PARP3 |
| | (Rubraca) | |
| myelodysplastic | ruxolitinib phosphate | JAK1 + JAK2 |
| and | (Jakafi) | |
| myeloproliferative | | |
| disorders | | |
| Breast | sacituzumab govitecan-hziy | TOP1 |
| | (Trodelvy) | |
| lymphoma | selinexor (Xpovio) | XPO1 |
| multiple | selinexor (Xpovio) | XPO1 |
| myeloma | | |
| lung | selpercatinib (Retevmo) | RET |
| solid tumors | selpercatinib (Retevmo) | RET |
| anywhere in | | |
| the body | | |
| thyroid | selpercatinib (Retevmo) | RET |
| plexiform | selumetinib sulfate | MEK1 + MEK2 |
| neurofibroma | (Koselugo) | |
| soft tissue | sirolimus protein-bound | MUT + mTOR |
| sarcoma | particles (Fyarro) | |
| skin | sonidegib (Odomzo) | SMO |
| kidney | sorafenib tosylate (Nexavar) | BRAF inhibitors , CRAF |
| | | inhibitors, FLT3 + PDGFRβ + |
| | | RET + VEGFR1 + VEGFR2 + |
| | | VEGFR3 + c-Kit |
| liver and bile | sorafenib tosylate (Nexavar) | BRAF + CRAF + FLT3 + |
| duct | | PDGFRβ + RET + VEGFR1 + |
| | | VEGFR2 + VEGFR3 + c-Kit |
| thyroid | sorafenib tosylate (Nexavar) | BRAF + CRAF + FLT3 + |
| | | PDGFRβ + RET + VEGFR1 + |
| | | VEGFR2 + VEGFR3 + c-Kit |
| lung | sotorasib (Lumakras) | KRAS G12C |
| Gastrointestinal | sunitinib malate (Sutent) | PDGFR + RTK + VEGFR |
| kidney | sunitinib malate (Sutent) | PDGFR + RTK + VEGFR |
| pancreatic | sunitinib malate (Sutent) | PDGFR + RTK + VEGFR |
| lymphoma | tafasitamab-cxix (Monjuvi) | CD19 |
| leukemia | tagraxofusp-erzs (Elzonris) | CD123 |
| Breast | talazoparib tosylate | PARP1, PARP2 |
| | (Talzenna) | |
| Breast | tamoxifen citrate (Soltamox) | ER |
| lymphoma | tazemetostat hydrobromide | EZH2 |
| | (Tazverik) | |
| soft tissue | tazemetostat hydrobromide | EZH2 |
| sarcoma | (Tazverik) | |
| skin | tebentafusp-tebn (Kimmtrak) | CD3 + gp100 |
| multiple | teclistamab-cqyv (Tecvayli) | CD3+ gp100 |
| myeloma | | |
| kidney | temsirolimus (Torisel) | CD3 + gp100 |
| lung | tepotinib hydrochloride | MET |
| | (Tepmetko) | |
| leukemia | tisagenlecleucel (Kymriah) | CD19 |
| lymphoma | tisagenlecleucel (Kymriah) | CD19 |
| Cervical | tisotumab vedotin-tftv | Tubulin, tissue factor |
| | (Tivdak) | |
| kidney | tivozanib hydrochloride | (VEGFR)-1, VEGFR-2 and |
| | (Fotivda) | VEGFR-3 , c-kit, and PDGFR β |
| Breast | toremifene (Fareston) | ER |
| Brain | trametinib (Mekinist) | MEK1 and MEK2 |
| lung | trametinib dimethyl | MEK1 and MEK2 |
| | sulfoxide (Mekinist) | |
| skin | trametinib dimethyl | MEK1 and MEK2 |
| | sulfoxide (Mekinist) | |
| solid tumors | trametinib dimethyl | MEK1 and MEK2 |
| anywhere in | sulfoxide (Mekinist) | |
| the body | | |
| thyroid | trametinib dimethyl | MEK1 and MEK2 |
| | sulfoxide (Mekinist) | |
| Breast | trastuzumab (Herceptin) | HER2 |
| esophageal | trastuzumab (Herceptin) | HER2 |
| stomach | trastuzumab (Herceptin) | HER2 |
| (gastric) | | |
| liver and bile | tremelimumab-actl (Imjudo) | CTLA-4, CD80 ,and CD86 |
| duct | | |
| lung | tremelimumab-actl (Imjudo) | CTLA-4, CD80 ,and CD86 |
| Breast | tucatinib (Tukysa) | HER2 |
| colorectal | tucatinib (Tukysa) | HER2 |
| thyroid | vandetanib (Caprelsa) | VEGFR-2 , EGFR, RET |
| skin | vemurafenib (Zelboraf) | BRAF, BRAF V600E, CRAF, |
| | | ARAF, SRMS, ACKI, MAP4K5, |
| | | FGR |
| leukemia | venetoclax (Venclexta) | BCL-2 |
| lymphoma | venetoclax (Venclexta) | BCL-2 |
| lymphoma | vorinostat (Zolinza) | HDAC1, HDAC2 and HDAC3 |
| | | (Class I) and HDAC6 (Class II) |
| leukemia | zanubrutinib (Brukinsa) | BTK |
| lymphoma | zanubrutinib (Brukinsa) | BTK |
| colorectal | ziv-aflibercept (Zaltrap) | PGF, VEGF-A |

In some embodiments, the methods described herein can be used to treat patients by (i) detecting one or more somatic mutations and/or cancer-related epigenetic signatures in the one or more target genes listed in Table 6; and (ii) administering the corresponding one or more drugs listed in Table 6. In some embodiments, these therapies may be used alone or in combination with other therapies to treat a disease.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA, such as cfDNA, isolated from a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject.

Thus, in some embodiments, quantities of each of one or more of a particular genetic and/or epigenetic signature (e.g., quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g., copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, genetic and/or epigenetic signatures, and/or cell types, are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

Therapeutic options for treating specific genetic-based diseases, disorders, or conditions, other than cancer, are generally well-known to those of ordinary skill in the art and will be apparent given the particular disease, disorder, or condition under consideration.

### IV. Kits

Also provided are kits, e.g., comprising the compositions as described herein. The kits can be useful in, or for use in, performing the methods as described herein. In some embodiments, the kit comprises target-specific probes that specifically bind to epigenetic and/or sequence-variable target region sets, wherein the target-specific probes of at least one epigenetic target region set bind to target regions that are differentially methylated in different immune cell types. In some such embodiments, the target-specific probes comprise a capture moiety. In some embodiments, the kit comprises a solid support linked to a binding partner of the capture moiety.

In some embodiments, a kit comprises an agent that recognizes methyl cytosine in DNA. In some such embodiments, the agent is an antibody or a methyl binding protein or methyl binding domain.

In some embodiments, a kit comprises reagents for a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity. The procedure that affects a first nucleobase of the DNA differently from a second nucleobase of the DNA may be any of the procedures described elsewhere herein.

In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1A, BRCA2, CCNE1, ESR1, RIT1, GATA3, MAP2K1, RHEB, ROS1, ARAF, MAP2K2, NFE2L2, RHOA, and NTRKI. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the can be found throughout and are applicable to the kit.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Determining an Epigenetic Consensus Sequence of DNA from a Patient Sample

The workflow described in this example is illustrated in FIG. 1A. A set of patient samples is analyzed by a blood-based NGS assay at Guardant Health (Redwood City, CA, USA) to detect the presence or absence of cancer. Double-stranded DNA is extracted from the blood of these patients. After concentrating the DNA, an end-repair step is performed, optionally with addition of an A tail. Next, adapters are added to the DNA by ligation to the 3′ ends thereof. These adapters contain non-unique molecular barcodes. The adapters may contain methylated bases so that they will be amplified post-bisulfite conversion.

After ligation, the DNA is washed and concentrated. The DNA is amplified using a methylation-preserving amplification method comprising DNMT1, such as linear amplification with thermocycling as described in Chang, et al., “DNA 5-Methylcytosine-Specific Amplification and Sequencing,” J. Am. Chem. Soc. 2020, 142(10):4539-4543. DNMT1 can be used at a concentration of about 100-10,000 U/mL, such as about 1,000 U/ml. The DNA can be amplified using common primers that contain methylated cytosines. Any Non-CpG ‘G’ in either primer sequence will be copied as unmethylated C and converted during base conversion, generating top and bottom strand sequences with non-complementary positions in the primer region. In one example, this limitation is overcome by using standard library adapter sequences and amplifying the DNA post-base conversion with a set of primers specific to all the possible conversion sequences at the library ends (four total: top and bottom strand-specific, forward and reverse primers). If Illumina sequencing is employed as a final step, custom sequencing primers can be used (e.g., a mix of two sequences for each read). Alternatively, the adapters can use alternative library adapter sequences and the standard Illumina sequencing primers can be appended during post-conversion amplification (as the 5′ tails of primers specific to the ligated adapter conversion products). For sequencing on other platforms, the post-base conversion PCR can be performed with a primer mix that adds the appropriate sequences for the ‘other’ platform (e.g., Ultima sequencing). Standard sequencing adapters can be used.

In a second example, a second ligation can be performed after methylation-preserving amplification (e.g., PCR using DNMT1) to append fully methylated NGS adapter sequences that are resistant to base conversion. In this case, for Illumina sequencing, the first ligation and PCR amplification should be performed with alternative Y-adapter sequences to avoid unintended multiple priming events in post-base conversion PCR. To avoid low complexity at the start of read 1 and the resulting low Illumina sequencing quality, the pre-conversion primers can have different 5′ end lengths and identities (e.g., random tails). Alternatively or in addition, the second ligation NGS adapters can have different lengths and identities (e.g., random) added bases at the ligation junction. For other platforms (e.g., Ultima) sequencing, the second ligation can contain ‘other’ platform sequencing adapters, and the first ligation adapters do not need to be altered.

The amplified DNA is then subjected to a procedure that modifies a first nucleobase of the DNA differently than a second nucleobase. For example, the DNA is treated with bisulfite in order to convert unmodified cytosines to uracils.

Following base conversion, the DNA can be enriched for nucleic acid molecules comprising sequences present in one or more epigenetic target region sets (such as specific hypermethylated and/or hypomethylated differentially methylated regions (DMRs)) and/or sequence variable target region sets (e.g., SNV/indel, fusion, CNV). The enriched DNA is subsequently amplified.

Following enrichment and amplification, the DNA is sequenced using NGS, and bioinformatics analyses of the NGS data are performed, wherein one or more components of each read is used to identify and group NGS reads into molecular families (e.g., DNA molecules), wherein the reads within a molecular family have (a) a common molecular barcode and (b) the same genomic start position, (c) the same genomic stop position, or (d) both (b) and (c). A consensus epigenetic sequence (methylation status) is identified for each cytosine in sequence of a given molecular family. A consensus genetic (i.e., nucleotide) sequence for each base may also be identified.

A tumor present/absent call, a tumor recurrence call, and/or a metastasis call is made based at least in part on (a) the presence and/or amount of methylated DNA molecules in regions that have been shown to be differentially methylated in cancer compared to normal cells, (b) the presence and/or amount of regions that have been shown to be differentially methylated in cell types that do not substantially contribute to cfDNA in healthy individuals, (c) the presence and/or amount of sequence-variable target regions that have been shown to differentiate cancer compared to normal cells, and/or (d) the presence and/or amount of sequence-variable target regions that have been shown to differentiate cell types that do not substantially contribute to DNA in healthy individuals from cell types that do so.

### Example 2: Determining an Epigenetic Consensus Sequence of DNA from a Patient Sample Using Rolling Circle Amplification

A set of patient samples is analyzed by a blood-based NGS assay at Guardant Health (Redwood City, CA, USA) to detect the presence or absence of cancer. Double-stranded DNA is extracted from the blood of these patients. After concentrating the DNA, an end-repair step is performed, optionally with addition of an A tail. Next, adapters are added to the DNA by ligation to the 3′ ends thereof. These adapters contain non-unique molecular barcodes. The adapters may contain methylated bases so that they will be amplified post-base conversion, although copies will contain methylated adapter sequences.

After ligation, the DNA is washed and concentrated. The DNA is linearly amplified using a methylation-preserving amplification method, wherein only a reverse primer is used so that DNA copies are synthesized only from the original template molecules (which have correct epigenetic marks), reducing error as compared to standard exponential amplifications (such as PCR with forward and reverse primers).

Alternatively, rolling circle amplification (RCA) may be performed wherein either hairpin DNA sequencing library adapters are ligated onto both sides of a DNA molecule or the DNA input molecules are circularized using intramolecular ligation. The hairpin adapter is designed with the same molecular barcodes and Y-adapter sequences as in a standard workflow using Y-adapters. The Y-portion ends are either directly connected to form hairpins or an additional connecting sequence or sequences are added. The RCA primer binds directly to the hairpin region. As the RCA product is synthesized, the methyltransferase (e.g., DNMT1) methylates the newly synthesized strand at C in CpG, opposite the 5mCpG in the template strand. The resulting product is a multimer of library amplicons, alternating between copies of the top and of the bottom strand of the molecule.

Following methylation-preserving amplification, the amplified DNA is then subjected to a procedure that modifies a first nucleobase of the DNA differently than a second nucleobase. For example, the DNA is treated with bisulfite in order to convert unmodified cytosines to uracils. Base conversion can be directly applied; if RCA is used in the earlier step, the RCA product can be amplified into monomer libraries with forward and reverse primers matching/hybridizing to the adapter ends (e.g., a mix of 4 primers, due to asymmetric sequences resulting from conversion, as discussed above).

Following base conversion, the DNA can be enriched for nucleic acid molecules comprising sequences present in one or more epigenetic target region sets (such as specific hypermethylated and/or hypomethylated differentially methylated regions (DMRs)) and/or sequence variable target region sets (e.g., SNV/indel, fusion, CNV). The enriched DNA is subsequently further amplified.

Following enrichment and amplification, the DNA is sequenced using NGS, and bioinformatics analyses of the NGS data are performed, wherein one or more components of each read is used to identify and group NGS reads into molecular families (e.g., DNA molecules), wherein the reads within a molecular family have (a) a common molecular barcode and (b) the same genomic start position, (c) the same genomic stop position, or (d) both (b) and (c). A consensus epigenetic sequence (methylation status) is identified for each cytosine in sequence of a given molecular family. A consensus genetic (i.e., nucleotide) sequence for each base may also be identified.

A tumor present/absent call, a tumor recurrence call, and/or a metastasis call is made based at least in part on (a) the presence and/or amount of methylated DNA molecules in regions that have been shown to be differentially methylated in cancer compared to normal cells, (b) the presence and/or amount of regions that have been shown to be differentially methylated in cell types that do not substantially contribute to cfDNA in healthy individuals, (c) the presence and/or amount of sequence-variable target regions that have been shown to differentiate cancer compared to normal cells, and/or (d) the presence and/or amount of sequence-variable target regions that have been shown to differentiate cell types that do not substantially contribute to DNA in healthy individuals from cell types that do so.

### Example 3: Determining an Epigenetic Consensus Sequence of DNA from a Patient Sample Using Methylation-Preserving Amplification and EM-Seq

The workflows of Examples 1-2 above are carried out as described, except bisulfite conversion is substituted with enzymatic-methyl (EM) base conversion. EM-seq is a non-destructive, base-converting method for single-base resolution methylation sequencing.

### Example 4: Determining an Epigenetic Consensus Sequence of DNA from a Patient Sample Using Methylation-Preserving Amplification and TAPS

The workflows of Examples 1-2 above are carried out as described, except bisulfite sequencing is substituted with TAPS (or another base conversion method that uniquely converts methylated cytosines, and not unmethylated cytosines).

### Example 5: Determining an Epigenetic Consensus Sequence of DNA from a Patient Sample Using Methylation-Preserving Amplification and SMRT Sequencing

The workflows of Examples 1-2 above are carried out as described, except that the DNA is not subjected to a procedure that modifies a first nucleobase of the DNA differently than a second nucleobase, and the sequencing comprises PacBio SMRT sequencing, which is used to identify methylated cytosine positions.

### Example 6: Determining an Epigenetic Consensus Sequence of DNA from a Patient Sample Using Methylation-Preserving Amplification and Nanopore Sequencing

The workflows of Examples 1-2 above are carried out as described, except that the DNA is not subjected to a procedure that modifies a first nucleobase of the DNA differently than a second nucleobase, and the sequencing comprises ONT nanopore sequencing, which is used to identify methylated cytosine positions.

### Example 7: Determining an Epigenetic Consensus Sequence of DNA from a Patient Sample Using Methylation-Preserving Amplification and 5-Letter or 6-Letter Sequencing

The workflows of Examples 1-4 above are carried out as described, except that the DNA is not subjected to a procedure that modifies a first nucleobase of the DNA differently than a second nucleobase, and the sequencing comprises 5-letter or 6-letter sequencing, which is used to identify methylated cytosine positions and/or hydroxymethylated cytosine positions.

1. (canceled)

2. A method of analyzing DNA, the method comprising:
(a) performing a methylation-preserving amplification of the DNA, wherein the DNA comprises barcodes;
(b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity;
(c) enriching for one or more sets of epigenetic target regions of DNA from the DNA, thereby providing enriched DNA, wherein the enriching comprises contacting the DNA with target-specific probes specific for the one or more sets of epigenetic target regions; and
(d) sequencing the DNA and determining an epigenetic consensus sequence of the DNA associated with at least a portion of the barcodes.

3. A method of analyzing DNA, the method comprising:
(a) performing a methylation-preserving amplification of the DNA, wherein the DNA comprises barcodes;
(b) enriching for one or more sets of epigenetic target regions of DNA from the DNA, thereby providing enriched DNA, wherein the enriching comprises contacting the DNA with target-specific probes specific for the one or more sets of epigenetic target regions;
(c) sequencing the enriched DNA in a modification-sensitive manner and determining an epigenetic consensus sequence of the enriched DNA associated with at least a portion of the barcodes.

4. A method of analyzing DNA, the method comprising:
(a) performing a linear, methylation-preserving amplification of the DNA; and
(b) sequencing the DNA in a modification-sensitive manner and determining an epigenetic consensus sequence of the DNA.

5. A method of analyzing DNA, the method comprising:
(a) performing a linear, methylation-preserving amplification of the DNA;
(b) subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity; and
(c) sequencing the DNA and determining an epigenetic consensus sequence of the DNA.

6. A method of analyzing DNA, the method comprising:
(a) performing a linear, methylation-preserving amplification of the DNA;
(b) enriching for one or more sets of epigenetic target regions of DNA from the DNA, thereby providing enriched DNA;
(c) sequencing the enriched DNA in a modification-sensitive manner and determining an epigenetic consensus sequence of the enriched DNA.

7. The method of claim 3, wherein the sequencing in a modification-sensitive manner comprises subjecting the DNA to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

8. The method of claim 2, wherein
(a) the DNA comprises barcodes,
(b) the method comprises ligating adapters comprising barcodes to the DNA prior to the sequencing;
(c) the method comprises ligating adapters comprising barcodes to the DNA prior to amplifying the DNA; and/or
(d) the method comprises ligating adapters comprising barcodes to the DNA prior to performing the methylation-preserving amplification of the DNA.

9.-10. (canceled)

12. A method of analyzing DNA, the method comprising:
(a) performing a methylation-preserving amplification of the DNA, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site, thereby providing amplified DNA;
(b) contacting the amplified DNA with a restriction enzyme that recognizes and cleaves the DNA at the restriction enzyme cleavage site in the adapter;
(c) before or after step (b), subjecting the amplified DNA to a procedure that affects a first nucleobase of the amplified DNA differently from a second nucleobase of the amplified DNA, wherein the first nucleobase is a modified or unmodified nucleobase, the second nucleobase is a modified or unmodified nucleobase different from the first nucleobase, and the first nucleobase and the second nucleobase have the same base pairing specificity;
(d) after steps (b) and (c), ligating supplemental adapters to the amplified DNA, optionally wherein the supplemental adapters do not comprise barcodes;
(e) after step (d), performing a uracil- and/or dihydrouracil-tolerant amplification of the DNA;
(f) after step (e), enriching for one or more sets of epigenetic target regions of DNA from the amplified DNA, thereby providing enriched DNA;
(g) optionally further amplifying the enriched DNA; and
(h) sequencing the enriched DNA and determining an epigenetic consensus sequence of the DNA associated with at least a portion of the barcodes.

13. The method of claim 12, wherein the uracil- and/or dihydrouracil-tolerant amplification of the DNA comprises PCR using a uracil- and/or dihydrouracil-tolerant DNA polymerase, and/or wherein the optional step of amplifying the enriched DNA (step (g)) is performed.

14. (canceled)

15. The method of claim 13, wherein the step of amplifying the enriched DNA further comprises differentially tagging the enriched DNA, optionally, wherein differentially tagging the enriched DNA comprises attaching one or more sample indices to the DNA.

16. (canceled)

17. The method of claim 2, wherein the barcodes do not comprise cytosines in a non-CpG context.

18. The method of claim 2, wherein the DNA comprises inserts and adapters comprising barcodes, at least one of the adapters further comprises a restriction enzyme cleavage site between a barcode and a portion of the adapter, and the barcode is located between the insert and the restriction enzyme cleavage site.

19. The method of claim 18, further comprising contacting the amplified DNA with a restriction enzyme that recognizes and cleaves the DNA at the restriction enzyme cleavage site in the adapter.

20. The method of claim 19, further comprising ligating supplemental adapters to the amplified DNA (a) after the step of contacting the amplified DNA with a restriction enzyme that recognizes and cleaves the DNA at the restriction enzyme cleavage site in the adapter, and (b) prior to or after a step of subjecting the amplified DNA to a procedure that affects a first nucleobase of the amplified DNA differently from a second nucleobase of the amplified DNA, wherein the first nucleobase is a modified or unmodified nucleobase, the second nucleobase is a modified or unmodified nucleobase different from the first nucleobase, and the first nucleobase and the second nucleobase have the same base pairing specificity; optionally wherein the supplemental adapters do not comprise barcodes.

21. The method of claim 20, further comprising performing a uracil- and/or dihydrouracil-tolerant amplification of the DNA, optionally wherein the uracil- and/or dihydrouracil-tolerant amplification of the DNA comprises PCR using a uracil- and/or dihydrouracil-tolerant DNA polymerase.

22.-23. (canceled)

24. The method of claim 19, wherein cleavage of the DNA by the restriction enzyme results in an overhang, optionally wherein the overhang is a single base overhang, further optionally wherein the single base overhang is a single base 5′-overhang.

25.-26. (canceled)

27. The method of claim 24, wherein the single base overhang is a T or an A.

28. (canceled)

29. The method of claim 2, wherein
(a) the methylation-preserving amplification comprises contacting the DNA with a methyltransferase, optionally wherein the methyltransferase preferentially methylates hemimethylated CpG and/or hemimethylated CpHpG, further optionally wherein the methyltransferase is DNMT1; and/or
(b) the methylation-preserving amplifying comprises one or more of polymerase chain reaction, linear amplification, rolling circle amplification, ligase chain reaction, strand displacement amplification, nucleic acid sequence-based amplification, and self-sustained sequence-based replication.

30.-42. (canceled)

43. The method of claim 2, wherein the method further comprises partitioning at least a portion of the DNA into a plurality of subsamples, comprising a first subsample and a second subsample, wherein the first subsample comprises DNA with a cytosine modification in a greater proportion than the second subsample, optionally wherein the partitioning at least a portion of the DNA into a plurality of subsamples comprises contacting the DNA with an agent that recognizes a modified cytosine in the DNA, wherein the first subsample comprises DNA with the modified cytosine in a greater proportion than the second subsample, further optionally wherein the agent that recognizes a modified nucleobase in the DNA is a methyl binding reagent.

44.-71. (canceled)

72. The method of claim 2, further comprising enriching for one or more sets of sequence-variable target regions of DNA from the DNA, wherein the enriching for one or more sets of sequence-variable target regions of DNA from the DNA comprises contacting the DNA with target-specific probes specific for the one or more sets of sequence-variable target regions, optionally wherein the epigenetic target region set comprises a hypermethylation variable target region set, a hypomethylation variable target region set, and/or a fragmentation variable target region set.

73-125. (canceled)
